(19) United States
(12) Patent Application Publication
(10) Pub. No.: US 2022/0259558 A1
(43) Pub. Date: Aug. 18, 2022

(54) METHODS FOR OBTAINING EYE FIELD PROGENITOR CELLS FROM HUMAN PLURIPOTENT STEM CELLS

(71) Applicant: Novo Nordisk A/S, Bagsvaerd (DK)

(72) Inventors: Juan Carlos Villaescusa Ramirez, Koebenhavn S (DK); Andreas Wrona, Koebenhavn NV (DK)

(21) Appl. No.: 17/610,961

(22) PCT Filed: May 14, 2020
(86) PCT No.: PCT/EP2020/063531

§ 371 Date: Nov. 12, 2021

(30) Foreign Application Priority Data
May 15, 2019 (EP) 19174780.7

(51) Int. Cl. C12N 5/079; C12N 5/0735

(52) U.S. Cl. CPC C12N 5/0621; C12N 5/0606; C12N 2501/15; C12N 2533/52; C12N 2501/727; C12N 2506/02; C12N 2506/45; C12N 2501/155

(57) ABSTRACT

The present invention relates to a method for obtaining eye field progenitor cells from hPSCs, which eye field progenitor cells are suitable for further differentiation into e.g. retinal pigmented epithelium cells and/or neural retina cells. The protocol provides a simple method with high yield of the cells of interest and facilitates translation into GMP compliance.

### TECHNICAL FIELD

The present invention relates to methods for efficiently obtaining eye field progenitor cells from human pluripotent stem cells (hPSCs), wherein said eye field progenitor cells are useful in further providing differentiated cells for the treatment of eye conditions. The present invention also relates to in vitro cell populations of eye field progenitor cells and their uses in the treatment of eye conditions. The protocol provides a simple and efficient method, while also facilitating translation into good manufacturing practice (GMP) compliance.

### INCORPORATION-BY-REFERENCE OF THE SEQUENCE LISTING

The present application is filed with a Sequence Listing in electronic form. The entire contents of the sequence listing are hereby incorporated by reference.

### BACKGROUND

The World Health Organization estimates that 314 million people have visual impairment worldwide, of whom 269 million have low vision and 45 million are blind (Resnikoff S., 2008). Some of these ophthalmologic disorders are cataracts, age-related macular degeneration (AMD), glaucoma, cornea blindness and Retinitis Pigmentosa (RP).

AMD is a disease that affects the macular region of the retina, causing progressive loss of central vision. The exact pathogenesis of AMD may not be fully elucidated, but it seems well-established that atrophy of the retinal pigment epithelium takes place, which is then followed by degeneration of essential retinal structures, such as neural retina cells thereby causing severe vision impairment. Currently, limited treatments are available and none of them regenerates the lost retina cells and repairs vision.

Cell implantation of e.g. healthy retinal pigment epithelium and neural retina cells for replacement therapy is thought to be a viable method of treatment of e.g. AMD to prevent blindness and even recover imperfect eyesight by delaying or restraining retinal degeneration, regenerating degenerated retina, and enhancing retinal functions.

Stem cells are a promising candidate for providing useful cell therapies for such cell implantation. The plasticity of pluripotent stem cells provides new possibilities for studying development and regeneration of the human eye to apply in different types of retinopathies, including but not limited to AMD and RP. However, obtaining cells such as retinal pigment epithelium (RPE) cells and neural retina (NR) cells for replacement therapy still remains a challenge. Over the last years, many protocols for the differentiation of hPSCs have been developed, which either recapitulate complete optic cup morphogenesis or aim at maximizing the generation of particular retinal cell subtypes. Protocols for the different cellular subtypes including RPE cells, and specific NR cell subtypes such as photoreceptors (PRs) and retinal ganglion cells (RGCs) have been described. The development towards the later stage eye progenitor cells is common and an intermediate cell type in the differentiation may be referred to as optic cup progenitor cells. Most available protocols require long differentiation periods, which in part is to arrive at the optic cup progenitor cells. A broader progenitor cell is referred here as early eye field progenitor cell, that comprises cells with the capability to generate different types of eye cells that include but are not limited to NR cells such as PRs and RGCs, RPE cells, lens cells and cornea cells, such as limbal stem stem cells. In many cases, the differentiation protocols also rely on a plurality of components such as growth factors, which may be expensive and/or difficult to bring into compliance with GMP. Moreover, many of the protocols suffer from limited cell specification and reproducibility.

It is an object of the present invention to overcome some of these challenges, in particular to provide shorter, more efficient and robust protocols for obtaining early eye field progenitor cells in a 2D setting, with the capacity to differentiate further into a variety of later stage, more mature, eye progenitor cells. It is another object of the present invention to provide a simple protocol that may facilitate translation into GMP compliance.

### SUMMARY

The aforementioned objects are achieved by the aspects of the present invention. In addition, the present invention may also solve further problems, which will be apparent from the disclosure of the exemplary embodiments.

An aspect of the present invention relates to an improved method for obtaining eye field progenitor cells from hPSCs, comprising the steps of culturing hPSCs, seeding the hPSCs on a substrate coated with a matrix, culturing the hPSCs in a cell culture medium to obtain differentiating cells, contacting the differentiating cells with an inhibitor of Small Mothers Against Decapentaplegic (SMAD) protein signaling pathway, and contacting the differentiating cells with BMPS, wherein the differentiating cells are allowed to differentiate into eye field progenitor cells.

This improved method facilitates a high number of cells suitable for further differentiation into later stage eye progenitors. Accordingly, another aspect of the present invention relates to an in vitro cell population of eye field progenitor cells, wherein a high percentage of the eye field progenitor cells co-express PAX6 and OTX2, and at least one of the group consisting of VSX2 and MITF, obtainable according to the methods of the present invention.

The inventors have shown that activating the bone morphogenetic protein (BMP) signaling pathway in stem cells effectively mature the differentiating cells into early eye field progenitor cells with the potential of further differentiating into a variety of more mature eye progenitor cells. In particular, the inventors have found that activating the BMP signaling pathway with BMP5 is very effective in differentiating the cells. Examples of such eye field progenitor cells, which the eye field progenitor cells may be further differentiated into more mature cells include but are not limited to RPE, NR cells such as PRs and RGCs, lens cells, and cornea cells, such as limbal stem cells (LSCs).

In one aspect of the present invention the eye field progenitor cells are RPE progenitor cells. Accordingly, the present invention also relates to an improved method for obtaining RPE progenitor cells from hPSCs, comprising the steps of culturing the hPSCs, seeding hPSCs on a substrate coated with a matrix, culturing the hPSCs in a cell culture medium to obtain differentiating cells, contacting the differentiating cells with an inhibitor of SMAD protein signaling, contacting the differentiating cells with BMP5, and contacting the differentiating cells with an inhibitor of GSK3, wherein the differentiating cells are allowed to differentiate into RPE progenitor cells.

In one aspect of the present invention the eye field progenitor cells are neural retina (NR) progenitor cells. Accordingly, the present invention also relates to an improved method for obtaining NR progenitor cells from hPSCs, comprising the steps of culturing the hPSCs, seeding hPSCs on a substrate coated with a matrix, culturing the hPSCs in a cell culture medium to obtain differentiating cells, contacting the differentiating cells with an inhibitor of SMAD protein signaling, contacting the differentiating cells with BMP5, wherein the differentiating cells are allowed to differentiate into RPE progenitor cells.

The inventors have further shown that the protocols according to the present invention provide a robust and efficient method for obtaining early eye field progenitor cells in a short period of time in a 2D setting. The protocols provide a high yield of the cells of interest and the method facilitates translation into GMP compliance.

### BRIEF DESCRIPTION OF DRAWINGS

FIG. 1 shows the effect of different laminins on the initial attachment of human embryonic stem cells (hESC) after 12 hours. Brightfield pictures shows how hESC that have been grown and maintained on LN-521 show excellent attachment to LN-332, in contrast to LN-111. LN-332 laminin shows positive effect on hESC attachment after single cell seeding. LN-332 can be used for further differentiation.

FIGS. 2A and 2B show the effect of human BMP5 and Activin A on the differentiation of hESC into VSX2 and MITF positive cells. Immunofluorescence showing conditions 1 and 2, without BMP5 or Activin A, with poor levels of MITF or VSX2. In contrast, the addition of BMP5 from day 12 (conditions 3 and 4), increase the number of cells positive for MITF and VSX2. The addition of Activin A from day 15, in combination with BMP5, has no clear additional effect. In contrast, the use of Activin A alone from day 12, or combination with BMP5 from day 15 (conditions 5 and 6), generates a lower number of cells positive for MITF and VSX2. In summary, only BMP5 shows a strong positive effect to generate MITF/VSX2 positive cells. The combination of IHH (Indian Hedgehog) and DKK2 (Dickkopf WNT Signaling Pathway Inhibitor 2) might help in the generation of MITF/VSX2 positive cells.

FIG. 3 shows that BMP5 induces the generation of double PAX6/OTX2 positive cells. An initial treatment with the small molecule GW788388, NOGGIN and Endo IWR1 for 12 days, followed by a treatment with BMP5, IHH and DKK2 generates high numbers of PAX6/OTX2 double positive cells addressed by immunofluorescence. DAPI is used for nuclear staining of all cells.

FIG. 4 shows the effect of BMP5 and the combination of BMP5 with a GSK3 inhibitor. The use of BMP5 induces the generation of MITF and VSX2 positive cells (second row), addressed by immunofluorescence, indicating the generation of neural retina progenitor cells. In contrast, the addition of the GSK3 inhibitor CHIR99021 (lower row) drastically blocks the expression of VSX2, and reinforces the expression of MITF and the generation of MITF positive cells with cobblestone morphology. This is indicative of RPE progenitor cells. First row, control without BMP5. DAPI is used for nuclear staining of all cells.

FIG. 5 shows RNA expression analyses. The graphs indicate the cycle threshold (CT) values from real-time polymerase chain reaction (PCR). Differentiated cells with BMP5 are collected at day 21 and RNA extracted, converted to cDNA and RNA expression analyses performed. hESC are used as comparison and CT values are inversely proportional to mRNA level. MITF, PAX6 and VSX2 expressions are upregulated in BMP5 differentiated cells compared to hESC.

FIG. 6 shows cell nuclei comparison between BMP5 differentiated cells and BMP5/CHIR99021. Notice the different nuclear organization of BMP5/CHIR99021 treated cells, indicating the epithelial morphology and the typical cobblestone morphology, indicative of RPE progenitor cells. DAPI is used for nuclear staining of all cells.

FIG. 7 shows that BMP5/CHIR99021 combination generates high number of MITF positive cells. The figure shows the high number of MITF positive cells and the high purity (more than 80%, immunofluorescence), when both BMP5 and CHIR99021 are used in combination. To the left, it is illustrated the high purity and homogeneity of the MITF positive cells with cobblestone morphology, indicative of RPE progenitor cells.

FIG. 8 shows that using the SMAD inhibitor RepSOX in combination with NOGGIN, and subsequent treatment with BMP5 and CHIR99021, our BMP5-based protocol generates eye field progenitor cells with an RPE progenitor cell identity. This is shown by the increased gene expression of PAX6, SIX3 and MITF together with immunofluorescence of OTX2 and PAX6 positive cells.

FIG. 9 shows the analysis of protein expression of hESC-derived RPE progenitor cells induced with GW788388, CHIR99021 and BMP5, by flow cytometry. More than 40% of the cells show co-expression of the markers PAX6/MITF.

FIG. 10 shows the analysis of protein expression of hESC-derived neural retina progenitor cells induced with GW788388 and BMP5, by flow cytometry. More than 50% of the cells show co-expression of the markers PAX6/VSX2.

FIG. 11 shows the percentages (table) of hESC-derived eye field progenitor cells with a RPE progenitor cell identity expressing indicated marker genes, analysed by single-cell RNA-sequencing. Induction of genes indicative of RPE and optic cup is seen in this table, whereas the cells do not express markers for the other germ layers (endoderm and mesoderm). Each Venn diagram shows expression patterns of cells co-expressing genes characteristic of RPE progenitors, PAX6/MITF/PMEL and PAX6/PMEL/SERPINF1 genes.

FIG. 12 shows Venn diagrams with the number of cells expressing markers of cornea and LSC. The percentage of triple positive cells for TP63/TFAP2B/S100A14 is 0.8%.

FIG. 13 shows the effect of different concentrations (0, 0.1, 200 and 1000 ng/ml) of BMP5 treatment on day 7-21 of differentiation together with CHIR99021 on day 12-21. RNA expression of RPE progenitor cell-related genes was quantified. Note that 200 ng/ml and 1000 ng/ml of BMP5 treatment promote the expression of RPE progenitor genes.

FIG. 14 shows the effect of different concentrations (0, 0.1, 200 and 1000 ng/ml) of BMP5 treatment on day 7-21. RNA expression of neural retina progenitor genes was quantified. 200 ng/ml and 1000 ng/ml of BMP5 treatment promote the expression of neural retina progenitor cell genes.

FIG. 15 shows comparison of different BMP isoforms on RPE progenitor cell gene expression. The effect of BMP5 is compared to that of BMP4, BMP7 and BMP4/7 heterodimer. BMP5 is superior to the other BMPs to induce the RPE progenitor genes indicated in the bar graph.

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Throughout this application the terms “method” and “protocol” when referring to processes for differentiating cells are used interchangeably.

As used herein, “a” or “an” or “the” can mean one or more than one. Unless otherwise indicated in the specification, terms presented in singular form also include the plural situation.

Also as used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

The term “about,” as used herein when referring to a measurable value such as an amount of cells, a compound or an agent of this invention, dose, temperature, and the like, is meant to encompass variations of 5%, 1%, 0.5%, or even 0.1% of the specified amount.

As used herein, the term “day” in reference to the protocols refers to a specific time for carrying out certain steps. In general, and unless otherwise stated, “day 0” refers to the initiation of the protocol, this be by for example but not limited to plating the stem cells or transferring the stem cells to an incubator or contacting the stem cells in their current cell culture medium with a compound prior to transfer of the stem cells. Typically, the initiation of the protocol will be by transferring undifferentiated stem cells to a different cell culture medium and/or container such as but not limited to by plating or incubating, and/or with the first contacting of the undifferentiated stem cells with a compound that affects the undifferentiated stem cells in such a way that a differentiation process is initiated.

When referring to “day X”, such as day 1, day 2 etc., it is relative to the initiation of the protocol at day 0. One of ordinary skill in the art will recognize that unless otherwise specified the exact time of the day for carrying out the step may vary. Accordingly, “day X” is meant to encompass a time span such as of +/−10 hours, +/−8 hours, +/−6 hours, +/−4 hours, +/−2 hours, or +/−1 hours.

As used herein, the phrase “from at about day X to at about day Y” refers to a day at which an event starts from. The phrase provides an interval of days on which the event may start from. For example, if “cells are contacted with a differentiating factor from at about day 3 to at about day 5” then this is to be construed as encompassing all the options: “the cells are contacted with a differentiating factor from about day 3”, “the cells are contacted with a differentiating factor from about day 4”, and “the cells are contacted with a differentiating factor from about day 5”. Accordingly, this phrase should not be construed as the event only occurring in the interval from day 3 to day 5. This applies mutatis mutandis to the phrase “to at about day X to at about day Y”.

Hereinafter, the methods according to the present invention are described in more detail by non-limiting embodiments and examples. Methods are provided for obtaining eye field progenitor cells, wherein the obtained cells are considered intermediates in further differentiation into cells such as mature RPE cells, NR cells, lens cells and corneal cells, from hPSCs, which again are being considered useful in providing a treatment of eye conditions such as cataracts, AMD, cornea blindness, glaucoma and RP.

According to the present invention the methods take offset in the use of stem cells.

### Stem Cells

By “stem cell” is to be understood as an undifferentiated cell having differentiation potency and proliferative capacity (particularly self-renewal competence), but maintaining differentiation potency. The stem cell includes subpopulations such as totipotent stem cell, pluripotent stem cell, multipotent stem cell, unipotent stem cell and the like according to the differentiation potency. Stem cells are classified by their developmental potential as: (1) totipotent, meaning able to give rise to all embryonic and extraembryonic cell types; (2) pluripotent, meaning able to give rise to all embryonic cell types; (3) multi-potent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multi-potent stem cells; and (5) unipotent, meaning able to give rise to a single cell lineage (e.g., spermatogenic stem cells). A pluripotent stem cell can be induced from fertilized egg, clone embryo, germ stem cell, stem cell in a tissue, somatic cell and the like. Examples of the pluripotent stem cell include embryonic stem cell (ES cell), EG cell (embryonic germ cell), induced pluripotent stem cell (iPS cell) and the like. In literature the “blastocyst-derived stem cell” are often referred to as embryonic stem cells, and more specifically human embryonic stem cells (hESC). The pluripotent stem cells used in the present invention can thus be embryonic stem cells prepared from blastocysts, as described in e.g. WO 03/055992 and WO 2007/042225, or be commercially available cells or cell lines. ES cell lines can also be derived from single blastomeres without the destruction of ex utero embryos and without affecting the clinical outcome (Chung et al. (2006) and Klimanskaya et al. (2006)). However, it is further envisaged that any hPSC can be used in the present invention, including differentiated adult cells which are reprogrammed to pluripotent cells by e.g. treating adult cells with certain transcription factors, such as but not limited to OCT4, SOX2, NANOG, and LIN28 as disclosed in Yu, et al. (2007); Takahashi et al. (2007) and Yu et al. (2009).

Muse cell (Multi-lineage differentiating stress enduring cell) obtained from mesenchymal stem cell (MSC), and GS cell produced from reproductive cell (e.g., testis) are also encompassed in the pluripotent stem cell. Induced pluripotent stem cells (also known as iPS cells or iPSCs) are a type of pluripotent stem cell that can be generated directly from adult cells. By the introduction of products of specific sets of pluripotency-associated genes adult cells can be converted into pluripotent stem cells. Embryonic stem cells can be produced by culturing an inner cell mass obtained without the destruction of the embryo. Embryonic stem cells are available from given organizations and are also commercially available.

In a most general aspect of the present invention is provided a method for obtaining eye field progenitor cells from hPSCs, comprising the steps of culturing hPSCs to obtain differentiating cells, and contacting the differentiating cells with BMPS, wherein the differentiating cells are allowed to differentiate into eye field progenitor cells. A more specific aspect relates to a method for obtaining eye field progenitor cells from hPSCs, comprising the steps of culturing hPSCs, seeding the hPSCs on a substrate coated with a matrix, culturing the hPSCs in a cell culture medium to obtain differentiating cells, contacting the differentiating cells with an inhibitor of SMAD protein signaling, and contacting the differentiating cells with BMPS, wherein the differentiating cells are allowed to differentiate into eye field progenitor cells.

The inventors found that the quality and yield of the cells obtained by the method according this aspect are high, and that the protocol can be based on compounds that easily translate into GMP compliance.

### Differentiation

As used herein “differentiate” or “differentiation” or “differentiating” refers to a process where cells progress from an undifferentiated state to a differentiated state, from an immature state to a less immature state or from an immature state to a mature state.

### Eye Field Progenitor Cells

The development towards the later stage eye progenitor cells is common and an intermediate cell type in the differentiation may be referred to as eye field progenitor cells. The general term “eye field progenitor cells” as used herein refers to an intermediate and transient group of progenitor cells, early in development, that includes multiple progenitor cells of different cell lineages of the eye, including but not limited to
a) optic cup progenitor cells which include the RPE progenitor cells and NR progenitor cells,
b) lens progenitor cells and
c) cornea progenitor cells, which include limbal stem cells (LSCs)

Eye field progenitor cells have the potential to differentiate into multiple eye cells, including but not limited to the RPE cells, all the different cell types of the NR, all the different cell types of the lens and all the different cell types of the cornea. The eye field progenitor cells are defined by the temporal expression of OTX2 and PAX6, together with other markers more specific of each cell linage.

As used herein “optic cup progenitor cells” refers to progenitor cells that are specified to further differentiate into NR cells and the RPE.

As used herein “cornea progenitor cells” refers to progenitor cells that are specified to further differentiate into the three cellular layers (the epithelium, stroma, and endothelium) and LSCs.

As used herein “lens progenitor cells” refers to progenitor cells that are specified to further differentiate into any cell type that forms the human lens.

As used herein “limbal stem cells (LSC)” refers to stem cells that have the ability to regenerate the entire corneal epithelium. LSC are also known as corneal epithelial stem cells,

As used herein a “neural retinal progenitor cell” is defined by the temporal expression of OTX2, PAX6 and VSX2 and MITF. The expression of MITF in NR progenitor cells is restricted to a very early phase in the differentiation process.

As used herein a “retinal pigmented epithelium (RPE) progenitor cell” is defined by the temporal expression of OTX2, PAX6, and MITF, cobblestone morphology, and the absence of VSX2.

“OTX2” as used herein refers to Orthodenticle Homeobox 2 gene, transcript or protein, and it is a marker of anterior brain structures during embryonic development including the eye field progenitor cells.

“PAX6” as used herein refers to “Paired Box 6” gene, transcript or protein and it is a marker of anterior brain structures during embryonic development including the eye field progenitor cells.

“SIX3” as used herein refers to “SIX Homeobox 3” gene, transcript or protein and it is a marker of eye field progenitor cells.

“SIX6” as used herein refers to “SIX Homeobox 6” gene, transcript or protein and it is a marker of eye field progenitor cells.

“MITF” as used herein refers to “Melanocyte Inducing Transcription Factor” gene, transcript or protein and it is a marker of RPE progenitor cells.

“PMEL17” or “PMEL” as used herein refers to “Premelanosome Protein” gene, transcript or protein and it is a marker of RPE progenitor and RPE mature cells.

“SERPINF1” as used herein refers to “Serpin Family F Member 1” gene, transcript or protein and it is a marker of RPE progenitor and RPE mature cells.

“TYR” as used herein refers to “Tyrosinase” gene, transcript or protein and it is a marker of RPE progenitor and RPE mature cells.

“VSX2” as used herein refers to “Visual System Homeobox 2” gene, transcript or protein, also known as CHX10, and it is a marker of Neural Retina progenitor cells.

“TP63” as used herein refers to “Tumor Protein P63” gene, transcript or protein, and it is a marker of LSC.

“S100A14” as used herein refers to “S100 Calcium Binding Protein A14” gene, transcript or protein, and it is a marker of LSC.

“TFAP2B” as used herein refers to “Transcription Factor AP-2 Beta” gene, transcript or protein, and it is a marker of LSC.

“ABCG2” as used herein refers to “ATP Binding Cassette Subfamily G Member 2” gene, transcript or protein, and it is a marker of LSC.

“NANOG” as used herein refers to “Nanog Homeobox” gene, transcript or protein, and it is a marker of pluripotent cells.

“POU5F1” as used herein refers to “POU Class 5 Homeobox 1” gene, transcript or protein, and it is a marker of pluripotent cells. “ZSCAN10” as used herein refers to “Zinc Finger And SCAN Domain Containing 10” gene, transcript or protein, and it is a marker of pluripotent cells.

“EOMES” as used herein refers to “Eomesodermin” gene, transcript or protein, and it is a marker of mesoderm lineage.

“SOX17” as used herein refers to “SRY-Box Transcription Factor 17” gene, transcript or protein, and it is a marker of endoderm lineage.

A skilled person will recognize that as the cells further differentiate one or more of these markers may change, such as but not limited to being up or down regulated. A skilled person will also recognize that the cells in question are not limited to the expression of only the aforementioned markers, but may also express other markers common to eye field progenitor cells.

In one embodiment, the present invention relates to a method for obtaining eye field progenitor cells, wherein at least 80% of the eye field progenitor cells express PAX6.

In one embodiment, the present invention relates to a method for obtaining eye field progenitor cells, wherein about 90% of the eye field progenitor cells express PAX6.

In one embodiment, the present invention relates to a method for obtaining eye field progenitor cells, wherein about 95% of the eye field progenitor cells express PAX6.

In one embodiment, the present invention relates to a method for obtaining eye field progenitor cells, wherein at least 40% of the eye field progenitor cells co-express PAX6 and OTX2.

In one embodiment, the present invention relates to a method for obtaining eye field progenitor cells, wherein at least 40% of the eye field progenitor cells co-express PAX6 and OTX2 and at least one of VSX2 and/or MITF.

In one embodiment, the present invention relates to a method for obtaining eye field progenitor cells, wherein at least 50%, 60%, 70%, 80%, or 90% of the eye field progenitor cells co-express PAX6 and OTX2, and at least 10%, 20%, 30%, 40%, 50% of the eye field progenitor cells further co-express VSX2 and/or MITF.

In one embodiment, the present invention relates to a method for obtaining eye field progenitor cells, wherein at least 50% of the eye field progenitor cells co-express PAX6 and VSX2.

In one embodiment, the present invention relates to a method for obtaining eye field progenitor cells, wherein at least 50% of the eye field progenitor cells co-express PAX6 and OTX2, and at least 20% of the eye field progenitor cells further co-express VSX2 and/or MITF.

In one embodiment, the present invention relates to a method for obtaining eye field progenitor cells, wherein at least 56% of the eye field progenitor cells co-express PAX6, OTX2 and SIX3.

In one embodiment, the present invention relates to a method for obtaining eye field progenitor cells, wherein at least 29% of the eye field progenitor cells co-express MITF, PMEL and SERPINF.

In one embodiment, the present invention relates to a method for obtaining eye field progenitor cells, wherein about 69% of the eye field progenitor cells co-express PMEL and SERPINF.

In one embodiment, the present invention relates to a method for obtaining eye field progenitor cells, wherein about 73% of the eye field progenitor cells express PMEL.

In one embodiment, the present invention relates to a method for obtaining eye field progenitor cells, wherein about 69% of the eye field progenitor cells express SERPINF.

In one embodiment, the present invention relates to a method for obtaining eye field progenitor cells, wherein at least 0.8% of said eye field progenitor cells co-express TP63, S100A14 and TFAP2B.

In one embodiment, the present invention relates to an in vitro cell population of eye field progenitor cells, wherein at least 80% of the eye field progenitor cells express PAX6.

In one embodiment, the present invention relates to an in vitro cell population of eye field progenitor cells, wherein about 90% of the eye field progenitor cells express PAX6.

In one embodiment, the present invention relates to an in vitro cell population of eye field progenitor cells, wherein about 95% of the eye field progenitor cells express PAX6.

In one embodiment, the present invention relates to an in vitro cell population of eye field progenitor cells, wherein at least 40% of the eye field progenitor cells co-express PAX6 and OTX2.

In one embodiment, the present invention relates to an in vitro cell population of eye field progenitor cells, wherein at least 40% of the eye field progenitor cells co-express PAX6 and OTX2 and at least one of VSX2 and/or MITF.

In one embodiment, the present invention relates to an in vitro cell population of eye field progenitor cells, wherein at least 50%, 60%, 70%, 80%, or 90% of the eye field progenitor cells co-express PAX6 and OTX2, and at least 10%, 20%, 30%, 40%, 50% of the eye field progenitor cells further co-express VSX2 and/or MITF.

In one embodiment, the present invention relates to an in vitro cell population of eye field progenitor cells, wherein at least 50% of the eye field progenitor cells co-express PAX6 and VSX2.

In one embodiment, the present invention relates to an in vitro cell population of eye field progenitor cells, wherein at least 50% of the eye field progenitor cells co-express PAX6 and OTX2, and at least 20% of the eye field progenitor cells further co-express VSX2 and/or MITF.

In one embodiment, the present invention relates to an in vitro cell population of eye field progenitor cells, wherein at least 56% of the eye field progenitor cells express SIX3.

In one embodiment, the present invention relates to an in vitro cell population of eye field progenitor cells, wherein about 73% of the eye field progenitor cells express PMEL.

In one embodiment, the present invention relates to an in vitro cell population of eye field progenitor cells, wherein about 69% of the eye field progenitor cells express SERPINF.

In one embodiment, the present invention relates to an in vitro cell population of eye field progenitor cells, wherein at least 0.8% of said eye field progenitor cells co-express TP63, S100A14 and TFAP2B.

In one embodiment, the present invention relates to a method for obtaining eye field progenitor cells, wherein said cells are RPE progenitor cells, NR cells or corneal cells.

In one embodiment, the present invention relates to a method for obtaining eye field progenitor cells, wherein said cells are RPE progenitor cells.

In one embodiment, the present invention relates to a method for obtaining eye field progenitor cells, wherein said cells are neural retina cells.

In one embodiment, the present invention relates to a method for obtaining eye field progenitor cells, wherein said cells are corneal cells.

The term “temporal expression of genes” used herein refers to the activation of genes within specific tissues or cells at specific times during development or differentiation.

The methods are defined by a series of steps. As used herein, the term “step” in relation to the method is to be understood as a stage, where something is undertaking and/or an action is performed. It will be understood by one of ordinary skill in the art when the steps to be performed and/or the steps undertaking are concurrent and/or successive and/or continuous.

In the step of culturing hESCs the cells may be obtained from any suitable source as referred to in the above. The step of seeding the hPSCs on a substrate coated with a matrix according to the method entails transferring the provided hPSCs. The term “seeding” is to be understood as the hPSCs being distributed to a suitable vessel. By the term “plating” is meant distributing the cells onto a suitable vessel with a substrate. A person skilled in the art will know the appropriate technique for transfer of undifferentiated cells onto a substrate. In an embodiment of the present invention, the hPSCs are plated with a density of from about 10,000 cells per cm² to about 100,000 cells per cm², preferably from about 20,000 cells per cm² to about 80,000 cells per cm², more preferably from about 30,000 cells per cm² to about 50,000 cells per cm², even more preferred about 40,000 cells per cm².

### Substrate

As used herein, the term “substrate” is to be understood as a surface onto which a coating may be provided. This may be but is not limited to well plates and beads. Typical substrates include but are not limited to cell culture treated multi-well plates, such as the Scientific™ Nunc™ Cell-Culture Treated multi-well plates. A person skilled in the art will readily acknowledge suitable substrates for culturing the cells. According to the present invention, the hPSCs provided are plated onto a substrate coated with a matrix.

By the term “matrix” is meant extracellular molecules that are responsible for interactions with cell surface receptors, thus regulating cell behavior such as adhesion, proliferation, migration and differentiation, or serve a mechanical supportive function. In one embodiment, the coating on the coated plates comprises laminin and/or fibronectin and/or vitronectin and/or collagen.

As used herein, the term “laminin” or “LN” in reference to coating on plates refers a heterotrimeric molecule consisting of three subunits termed alpha, beta and gamma chains. The references herein are made to human laminin. Five kinds of a chains (alpha 1 to alpha 5), three kinds of beta chains (beta 1 to beta 3) and three kinds of gamma chains (gamma1 to gamma3) are known, and various combinations of these chains give rise to at least 12 kinds of laminin isoforms. For example, “laminin alpha 5 beta1 gamma1” is herein referred to as “laminin-511” or “LN-511”. The same will apply to other isoforms. By “fragment thereof” when referring to laminin is meant part of the intact laminin. For instance, it has been found that the E8 fragment of laminin-511 strongly adhere to human embryonic stem cells. Laminins and fragments thereof are commercially available from companies such as Biolamina AB or Nippi Inc. Non-limiting examples of laminins include LN-111, LN-423, LN-523, LN-511, LN-521 and LN-332 or fragments thereof.

In one embodiment the matrix used in the method of the present invention is a laminin or fragment thereof, selected from the group consisting of LN-111, LN-423, LN-523, LN-511, LN-521 and LN-332.

In one embodiment the matrix used in the method of the present invention is LN-423 or a fragment thereof.

In one embodiment the matrix used in the method of the present invention is LN-511, or a fragment thereof.

In one embodiment the matrix used in the method of the present invention is LN-521, or a fragment thereof.

In one embodiment the matrix used in the method of the present invention is LN-332 or a fragment thereof.

As used herein, the term “fibronectin” in reference to coating on plates refers to a high-molecular weight (˜440 kDa) glycoprotein of the extracellular matrix that binds to membrane-spanning receptor proteins called integrins Similar to integrins, fibronectin binds extracellular matrix components such as collagen, fibrin, and heparan sulfate proteoglycans (e.g. syndecans). As used herein, the term “vitronectin” in reference to coating on plates refers to a glycoprotein of the hemopexin family which is abundantly found in serum, the extracellular matrix and bone. As used herein, the term “collagen” in reference to coating on plates refers to a structural protein in the extracellular space in the various connective tissues in animal bodies. As the main component of connective tissue, it is the most abundant protein in mammals making 25% to 35% of the whole-body protein content. Collagen consists of amino acids wound together to form triple-helices to form of elongated fibrils.

In a preferred embodiment, the matrix coated onto the substrate comprises a laminin or a fragment thereof, preferably selected from the group consisting of laminin-511 and laminin-332.

In another embodiment, the laminin or fragment thereof is a combination of laminin-511 and laminin-332.

In one embodiment the matrix comprises laminin-511 and/or laminin-332 and one or more further laminin(s).

In another embodiment, the laminin or fragment thereof is laminin-332. In one embodiment, the laminin is an intact laminin protein.

In another embodiment, the laminin is a fragment of the intact laminin protein. In a further embodiment, the concentration of the laminin is from about 0.01 μg/cm² to about 50 μg/cm², preferably from about 0.1 μg/cm² to about 25 μg/cm², more preferably from about 0.1 μg/cm² to 10 μg/cm², more preferably from about 0.1 μg/cm² to about 5, more preferably from about 0.25 μg/cm² to about 1 μg/cm², even more preferably about 0.5 μg/cm².

The step of culturing is to be understood as a process by which the stem cells are grown under controlled conditions, generally outside their natural environment. The term “culturing” is to be understood as a continuous procedure, which is employed throughout the method in order to maintain the viability of the cells at their various stages. After the cells of interest have been isolated from, for example but not limited to, living tissue or embryo, they are subsequently maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO₂, O₂), and regulates the physio-chemical environment (pH buffer, osmotic pressure, temperature).

In a one embodiment, the steps of seeding and culturing occur simultaneously, i.e. the hPSCs are plated on a substrate comprising a cell culture medium. In an embodiment, the differentiation process is immediately initiated at the seeding and culturing. The culturing step alone is not to be construed as a step culturing differentiating cells, but merely culturing the stem cells is a prerequisite for the further steps to obtain differentiating cells. Notwithstanding, the hPSCs as cultured are now referred to as differentiating cells. As used herein, the term “differentiating cells” refers to cells to undergo or undergoing a process by which the cells differentiate from one cell type (e.g. a multipotent, totipotent or pluripotent differentiable cell) to another cell type such as a target differentiated cell, which according to the present invention is an eye field progenitor cell. Even though the cell may have developed into a cell type that can be classified, the term “differentiating cell” may still be used.

In one embodiment, the cell culture medium in the culturing step is a first cell culture medium and wherein at least part of the cell culture medium is subsequently replaced with a second cell culture medium. Accordingly, in one embodiment, the cell culture medium at day 0 is a first cell culture medium and wherein at least part of the cell culture medium is replaced with a second cell culture medium from about day 1. In a preferred embodiment, the cell culture medium in the seeding step at day 0 is a first cell culture medium and wherein the first cell culture medium is substantially replaced with a second cell culture medium from about day 1.

### ROCK Inhibitor

Rho-associated coiled-coil containing kinases (ROCK) is an effector of the RhoA small GTPase and belongs to the AGC family of serine/threonine kinases. ROCK kinases have many functions including cell contraction, migration, apoptosis, survival, and proliferation. IRho-associated, coiled-coil containing protein kinase ROCK inhibitors are a series of compounds that target and inhibit rho kinase. As used herein, “Y-27632” refers to trans-4-(1-Aminoethyl)-N-(4-Pyridyl) cyclohexanecarboxamide dihydrochloride with CAS no. 129830-38-2.

In one embodiment, the first cell culture medium comprises a ROCK inhibitor. In one embodiment the ROCK inhibitor is Y-27632 or Tiger.

In one embodiment, the first cell culture medium comprises said Rock inhibitor in the concentration range of 0.1-30 μM.

In one embodiment, the first cell culture medium comprises said Rock inhibitor in the concentration range of 1-20 μM.

In one embodiment, the first cell culture medium comprises said Rock inhibitor in the concentration of about 10 μM.

### Culture Media

Typically, the stem cells will be provided in a cell culture medium, which is suitable for viability in their current state of development. Providing the stem cells for culturing typically implies a transfer of the stem cells into a different environment such as by seeding onto a new substrate or suspending in an incubator. One of ordinary skill in the art will readily recognize that stem cells are fragile to such transfer and the procedure require diligence and that maintaining the stem cells in the origin cell culture medium may facilitate a more sustainable transfer of the cells before replacing the cell culture medium with another cell culture medium more suitable for the differentiation process. In one embodiment of the method, the cell culture medium in the seeding step at day 0 is a first cell culture medium and at least part of the cell culture medium is replaced with a second cell culture medium from day 1. As used herein, the term “replacing” in reference to cell culture medium, first cell culture medium, and second cell culture medium means a procedure, wherein an amount of cell culture medium is taken out by suitable means, and, optionally, a substantially equal amount of cell culture medium is added so that the total volume of cell culture medium substantially remains the same. By “removing the first cell culture medium” is to be understood as after a first removal and addition of the second cell culture medium then any subsequent replacement will be a replacement of a mixture of the first and second cell culture medium, the mixture being in the ratio corresponding to the amounts removed and added. Accordingly, in a sequential removal, the first cell culture medium will be continuously diluted by the second cell culture medium and by repeating this procedure the cell culture medium eventually will be substantially free of the first cell culture medium. In a preferred embodiment, the first cell culture medium is substantially replaced with a second cell culture medium at about day 1.

In a further embodiment the first cell culture medium is chemically defined and xeno-free. As used herein, the term “chemically defined” in reference to a cell culture medium means a growth medium suitable for the in vitro cell culture of human or animal cells in which all of the chemical components are known. The chemically defined media require that all of the components must be identified and have their exact concentrations known. As used herein, the terms “xeno-free” and “animal-free” may be used interchangeably and according to the present invention mean preferably completely devoid of any animal-derived components. In a preferred embodiment, the cell culture medium is also feeder-free. The terms “feeder-free” and “feeder cell-free” may be used interchangeably and refer to the culturing system being devoid of human and animal cells which may be otherwise present for the purpose of nourishing the cultured stem cells, i.e. the feeder cells supply metabolites to the stem cells they support, but are not the cells intended for growth or division.

Even though the present inventors prefer a chemically defined, “xeno-free” and “feeder cell-free” cell culturing environment, regulatory bodies may approve medicinal products and treatments based on the methods according to the present invention without fully complying with such standard. The present inventors endeavor to adhere to the highest standards of GMP and good tissue practices (GTP). However, the present invention should not be construed as limited to such standards. A person skilled in the art will readily acknowledge that the present invention may be carried out without adhering to such high standards.

In a one embodiment the first cell culture medium may be any suitable cell culture medium which supports viability of the stem cells upon transfer to the substrate. Such cell culture media are commercially available and could for instance be Nutristem®, such as Nutristem® hPSC XF Medium for iPS and ES Stem Cells. Accordingly, in one embodiment the Nutristem®, such as Nutristem® hPSC XF Medium for iPS and ES Stem Cells.

In one embodiment the second cell culture medium is chemically defined and xeno-free. In a further embodiment, the second cell culture medium is also feeder-free. In one embodiment the second cell culture medium comprises GMEM (Glasgow's Modified Essential Medium) or DMEM/F12 (Dulbecco's Modified Eagle Medium/Ham's F-12 Medium). Similar media may work equally well and are readily available for purchase. In a further embodiment the GMEM or DMEM/F12 is supplemented with N2 and/or B27. In one embodiment, the concentration of B27 from about 0.1% (v/v) to about 5% (v/v), preferably from about 0.5% (v/v) to about 2.5% (v/v), even more preferred about 2% (v/v). In one embodiment, the concentration of N2 from about 0.1% (v/v) to about 5% (v/v), preferably from about 0.5% (v/v) to about 2.5% (v/v), even more preferred about 1% (v/v).

In one embodiment, the differentiating cells are contacted with an inhibitor of SMAD protein signaling.

As used herein, by the term “contacting” in reference to culturing cells is meant exposing the cells to e.g. a specific compound by placing the specific compound in a location that will allow it to touch the cell in order to produce “contacted” cells. The contacting may be accomplished using any suitable means. A non-limiting example of contacting is by adding the compound to a cell culture medium of the cells. The contacting of the cells is assumed to occur as long as the cells and specific compound are in proximity, e.g. the compound is present in a suitable concentration in the cell culture medium.

As used herein, the term “inhibitor” in reference to inhibiting a signaling target or a signaling target pathway refers to a compound that interferes with (i.e. reduces or eliminates or suppresses) a resulting target molecule or target compound or target process, such as a particular differentiation outcome, (for example, suppresses an active signaling pathway promoting a default cell type differentiation, thereby inducing differentiation into a non-default cell type) when compared to an untreated cell or a cell treated with a compound that does not inhibit a treated cell or tissue.

### Inhibitor of the Small Mothers Against Decapentaplegic (SMAD) Protein Signaling Pathway

As used herein “inhibitor of the Small Mothers Against Decapentaplegic (SMAD) protein signaling pathway” refers to a compound that specifically inhibits the Small Mothers Against Decapentaplegic (SMAD) protein signaling pathway. Examples of inhibitor of Small Mothers Against Decapentaplegic (SMAD) protein signaling may be selected from the group comprising GW788388, LDN-193189, LY2157299, LY364947, NOGGIN, RepSOX, SB431542, and TEW-7197.

As used herein, “GW788388” denotes a small molecule chemical name N-(oxan-4-yl)-4-[4-(5-pyridin-2-yl-1H-pyrazol-4-yl)pyridin-2-yl]benzamide and CAS no: 452342-67-5.

As used herein, “LDN-193189” denotes a compound with the IUPAC name 4-(6-(4-(Piperazin-1-yl)phenyl)pyrazolo[1,5-a]pyrimidin-3-yl)quinoline and CAS no: 1062368-24-4.

As used herein, “LY2157299” denotes a small molecule, which is potent TGFβ receptor I (TGFβRI) inhibitor with alternative name Galunisertib and chemical name 4-[2-(6-methylpyridin-2-yl)-5,6-dihydro-4H-pyrrolo[1,2-b]pyrazol-3-yl]quinoline-6-carboxamide, and CAS no: 700874-72-2.

As used herein, “LY364947” denotes compound with the IUPAC name 4-[3-(2-pyridinyl)-1H-pyrazol-4-yl]-quinoline and CAS no: 396129-53-6.

As used herein, “NOGGIN” denotes a secreted homodimeric glycoprotein that binds to and inactivates members of the transforming growth factor-beta (TGF-β) superfamily of signaling proteins, such as bone morphogenetic protein-4 (B MP 4). NOGGIN is typically a 65 kDa protein expressed in human cells as a glycosylated, disulfide-linked dimer.

As used herein, “RepSOX” denotes a small molecule, which is a potent and selective inhibitor of TGF-βR1 with alternative names E-616452, SJN 2511, ALK5 Inhibitor II, and chemical name 2-(3-(6-Methylpyridine-2-yl)-1H-pyrazol-4-yl)-1,5-naphthyridine, and CAS no: 446859-33-2.

As used herein, “SB431542” denotes a compound with the chemical name 4-[4-(1,3-benzodioxol-5-yl)-5-(2-pyridinyl)-1H-imidazol-2-yl]benzamide and CAS no: 301836-41-9.

As used herein, “TEW-7197” denotes a small molecule with alternative name Vactosertib and chemical name 2-fluoro-N-[[5-(6-methylpyridin-2-yl)-4-([1,2,4]triazolo[1,5-a]pyridin-6-yl)-1H-imidazol-2-yl]methyl]aniline and CAS no: 1352608-82-2.

In one embodiment, the present invention relates to a method for obtaining eye field progenitor cells, wherein said method comprises at least one SMAD inhibitor.

In one embodiment, the present invention relates to a method for obtaining eye field progenitor cells, wherein said method comprises two SMAD inhibitors.

In one embodiment, the present invention relates to a method for obtaining eye field progenitor cells, wherein said method comprises a single SMAD inhibitor.

In one embodiment, the differentiating cells are contacted with an inhibitor of Small Mothers Against Decapentaplegic (SMAD) protein signaling selected from the group consisting of GW788388, LDN-193189, LY2157299, LY364947, NOGGIN, RepSOX, SB431542, and TEW-7197.

In another embodiment, the differentiating cells are contacted with an inhibitor of SMAD protein signaling selected from the group consisting of GW788388 and/or RepSOX.

In one embodiment, the present invention relates to a method for obtaining eye field progenitor cells, wherein said method comprises GW788388 and/or RepSOX.

In one embodiment, the present invention relates to a method for obtaining eye field progenitor cells, wherein said method comprises GW788388.

In one embodiment, the present invention relates to a method for obtaining eye field progenitor cells, wherein said method comprises RepSOX.

The inventors identified these inhibitors of SMAD protein signaling as providing an effective and robust initiation of differentiation into eye field progenitor cells. The small molecules furthermore facilitate translation into GMP compliance.

In one embodiment the inhibitor of SMAD protein signaling is GW788388.

In a further embodiment thereof, the inhibitor of SMAD protein signaling is GW788388 in a concentration of from about 0.1 ng/ml to about 1,000 ng/ml, preferably from about 5 ng/ml to about 1,000 ng/ml, more preferably from about 5 ng/ml to about 500 ng/ml, more preferably from about 5 ng/ml to about 250 ng/ml, more preferably from about 5 ng/ml to about 100 ng/ml, more preferably from about 5 ng/ml to about 50 ng/ml. In one embodiment, the concentration is from about 5 ng/ml to about 20 ng/ml, such as about 10 ng/ml.

In one embodiment, the inhibitor of SMAD protein signaling is RepSOX.

In another embodiment, the inhibitor of SMAD protein signaling is RepSOX in a concentration of from about 0.25 μM to about 200 μM, preferably from about 10 μM to about 150 μM, more preferably from about 15 μM to about 100 μM, even more preferably from about 20 μM to about 75 μM.

In one embodiment, the differentiating cells are contacted with an inhibitor of SMAD protein signaling pathway from day 0. It follows that in such an embodiment the inhibitor of the SMAD protein signaling pathway is added to the first cell culture medium.

In a preferred embodiment, the differentiating cells are contacted with the inhibitor of SMAD protein signaling from day 0 to day 15, day 14, day 13, day 12, day 11, or day 10, preferably from day 0 to day 12. It follows that in such an embodiment the inhibitor of the SMAD protein signaling pathway is also added to the second cell culture medium.

In one embodiment, the inhibitor of SMAD protein signaling comprises only one compound.

In one embodiment, the inhibitor of SMAD protein signaling comprises more than one compound, such as but not limited to a combination of the aforementioned inhibitors of SMAD protein signaling. A person skilled in the art will recognize that the concentration of the individual inhibitors of SMAD protein signaling may need to be adjusted accordingly to obtain similar effect as one would with the individual inhibitors.

The present inventors have found that the hPSCs may be differentiated into eye field progenitor cells with a protocol exposing the cells to only one inhibitor of SMAD protein signaling.

In one embodiment, the differentiating cells are contacted with only one inhibitor of SMAD protein signaling. This simplifies the differentiation protocol, reduces costs, and further facilitate translation into GMP compliance.

The differentiating cells are contacted with BMPS or an analog thereof. As used herein the terms “analog” and “variant” may be used interchangeably and are used to define peptides or proteins that differ from the native or reference peptide or protein by virtue of one or more amino acid changes.

### BMP5

As used herein, “BMP5” refers to human bone morphogenetic protein 5 or an analog thereof. BMP5 is an activator of the BMP signaling pathway, a protein that in humans is encoded by the BMP5 gene and is member of the TGFβ superfamily. The human BMP5 (bone morphogenetic protein 5) isoform 1 preproprotein is identified by SEQ ID NO: 1. A person skilled in the art will readily recognize that variants of this sequence may exist such as but not limited to at various stages of the protein synthesis and maturation, and that such variants may work equally as well as BMP5 identified by SEQ ID NO: 1.

In one embodiment, BMP5 is identified by SEQ ID NO: 1.

In one embodiment, the differentiating cells are contacted with BMP5 (SEQ ID NO: 1) or an analog thereof, wherein the analog thereof is an effective activator of the bone morphogenetic protein (BMP) signaling pathway. The present inventors have found that BMP5 is an effective activator of the bone morphogenetic protein (BMP) signaling pathway, that enables fast and effective differentiation into eye field progenitor cells.

In one embodiment, the differentiating cells are contacted with BMP5 (SEQ ID NO: 1) or an analog thereof, wherein the analog has at least 50% identity with BMP5 identified by SEQ ID NO: 1.

In one embodiment, the analog of BMP5 has at least 50%, 60%, 70%, 80%, 90%, 91%, 92%, 93%, 94%, 95%, 96%, 97%, 98%, or 99% identity with BMP5 identified by SEQ ID NO: 1.

In a preferred embodiment, the differentiating cells are contacted with an effective amount of BMP5 (SEQ ID NO: 1) or an analog thereof. By the term “effective amount” is meant contacting the differentiating cells with a concentration of BMP5 or an analog thereof, wherein the activity of BMP5 or the analog thereof is sufficiently high to promote the further differentiation of the differentiating cells towards an eye field progenitor fate. A skilled person will acknowledge that the activity of BMP5 and analogs thereof may vary. This may even be the case for seemingly identical products provided by different vendors. Accordingly, in one embodiment the activity (ED₅₀) of BMP5is from about 0.1 μg/ml to about 2 μg/ml, preferably from about 0.15 μg/ml to about 1.5 μg/ml, more preferably from about 0.2 μg/ml to about 1.3 μg/ml, even more preferably from about 0.21 μg/ml to about 1.2 μg/ml. In one embodiment, this activity may be correlated with the concentration of BMP5. The activity as referred to can be measured as described in “Activity Measured by its ability to induce alkaline phosphatase production by ATDCS mouse chondrogenic cells”, Nakamura, K. et al. (1999) Exp. Cell Res. 250:351.

In one embodiment, the concentration of BMP5 is at least about 0,1, 1, 5, 10, 20, 30, 40, 50, 60, 70, 80, 90, 100, 110, 120, 130, 140, 150, 160, 170, 180, 190, 200, 300, 400, 500, 1000, 2000 or 2500 ng/ml, preferably at least about 150 ng/ml, more preferably at least about 180 ng/ml.

In one embodiment the concentration of BMP5 is below about 1000, 900, 800, 700, 600, or 500 ng/ml, preferably below about 450 ng/ml.

In one embodiment the concentration of BMP5 is in the range of about 0.1 ng/ml to about 2000 ng/ml.

In one embodiment the concentration of BMP5 is in the range of about 0.1 ng/ml to about 1000 ng/ml.

In one embodiment the concentration of BMP5 is in the range of about 10 ng/ml to about 300 ng/ml.

In one embodiment the concentration of BMP5 is in the range of about 100 ng/ml to about 300 ng/ml.

In one embodiment the concentration of BMP5 is in the range of about 150 ng/ml to about 250 ng/ml.

In one embodiment the concentration of BMP5 is about 200 ng/ml.

In one embodiment the concentration of BMP5 is about 1000 ng/ml.

In one embodiment the concentration of BMP5 is in the range of about 200 ng/ml to about 1000 ng/ml.

In one embodiment the concentration of BMP5 is in the range of about 150 ng/ml to about 1100 ng/ml.

In one embodiment the concentration of BMP5 is in the range of about 150 ng/ml to about 500 ng/ml.

In one embodiment the concentration of BMP5 is in the range of about 150 ng/ml to about 250 ng/ml.

In one embodiment, in the step of contacting the differentiating cells with BMP5, the concentration of BMP5 is from about 100 ng/ml to about 600 ng/ml, preferably from about 150 ng/ml to about 550 ng/ml, more preferably from about 200 ng/ml to about 500 ng/ml, more preferably from about 200 ng/ml to about 400 ng/ml.

In a further embodiment the concentration of BMP5is from about 350 ng/ml to about 450 ng/ml, preferably from about 360 ng/ml to about 440 ng/ml, more preferably from about 370 ng/ml to about 430 ng/ml, more preferably from about 380 ng/ml to about 420 ng/ml, more preferably from about 390 ng/ml to about 410 ng/ml, even more preferably about 400 ng/ml.

In one embodiment, the differentiating cells are contacted with BMP5 from at about day 5 to at about day 14, preferably from at about day 6 to at about day 13, more preferably from at about day 6 to at about day 12, more preferably from at about day 6 to at about day 11, more preferably from at about day 6 to at about day 10, more preferably from at about day 6 to at about day 9, more preferably from at about day 6 to at about day 8, even more preferably from at about day 7.

In one embodiment, the differentiating cells are contacted with BMP5, wherein the differentiating cells are allowed to differentiate into eye field progenitor cells until about day 30, 29, 28, 27, 26, 25, 24, 23, 22, 21, 20, or 19, preferably until about day 20 to day 22, even more preferably until about day 21.

In one embodiment, the differentiating cells are contacted with BMP5 from about day 5 to about day 30, from about day 5 to about day 29, from about day 5 to about day 28, from about day 5 to about day 27, preferably from about day 6 to about day 26, more preferably from about day 6 to about day 25, more preferably from about day 6 to about day 24, more preferably from about day 6 to about day 23, more preferably from about day 6 to about day 22, more preferably from about day 6 to about day 21, even more preferably from about day 7 to about day 21.

Allowing the differentiating cells to differentiate is not to be construed as a separate final step to be performed. One of ordinary skill in the art will readily appreciate that as used herein the terms “differentiate” and “differentiation” refer to the process wherein cells progress from an undifferentiated state to a differentiated state, from an immature state to a less immature state or from an immature state to a mature state, which occurs continuously as the method is performed. This is for example but not limited to hPSCs differentiating into eye field progenitor cells. Changes in cell interaction and maturation occur as cells lose markers of undifferentiated cells or gain markers of differentiated cells. Loss or gain of a single marker can indicate that a cell has “matured or fully differentiated”.

One of ordinary skill in the art will be able to determine when the differentiating cells have matured into eye field progenitor cells based on specific markers. Accordingly, in some embodiments, the differentiating cells are allowed to differentiate into eye field progenitor cells for about 17 day to 40 days, preferably for about 18 days to 30 days, more preferably for about 19 days to 25 days, more preferably for about 19 days to 23 days, more preferably for about 20 days to 22 days, even more preferably for about 21 days, starting from day 0.

In one embodiment, the differentiating cells are contacted with an inhibitor of SMAD protein signaling from about day 0 to about day 12, and the differentiating cells are contacted with BMP5 from about day 7, wherein the differentiating cells are allowed to differentiate into eye field progenitor cells until about day 21.

Although the differentiating cells may be considered as fully differentiated into eye field progenitor cells, a further differentiation may proceed towards further specified or matured progenitor cells, such as but not limited to RPE cells and NR cells, wherein additional factors and/or conditions may be employed. It is specifically an object of the present invention to provide cells that may be further differentiated into more mature progenitor cells or fully matured cells for use in e.g. a treatment of an eye condition.

The hPSCs are allowed to differentiate into eye field progenitor cells. In one embodiment, at least 10%, at least 20%, at least 30%, at least 40%, at least 50%, at least 60%, at least 70%, at least 80%, or at least 90% of the eye field progenitor cells co-express PAX6 and OTX2, and one or more of VSX2 and MITF. In a further embodiment, at least 50%, at least 60%, at least 70%, at least 80%, or at least 90% of the eye field progenitor cells co-express PAX6 and OTX2, and at least 10%, at least 20%, at least 30%, at least 40%, at least 50% of the eye field progenitor cells further co-express one or more of VSX2 and MITF.

In one embodiment the method comprises a further step, wherein the differentiating cells are contacted with an inhibitor of the WNT/β-catenin pathway. As used herein, the term “WNT/β-catenin pathway” refers a group of signal transduction pathways which begin with proteins that pass signals into a cell through cell surface receptors. Wnt is an acronym in the field of genetics that stands for ‘Wingless/Integrated’.

In one embodiment, the differentiating cells are contacted with an inhibitor of the WNT/β-catenin pathway from about day 2 to about day 15, preferably from about day 3 to about day 14, more preferably from about day 3 to about day 13, even more preferably from about day 3 to about day 12. In one embodiment, the inhibitor of the WNT/β-catenin pathway is Endo IWR 1. In a further embodiment, the concentration of Endo IWR 1 is from about 0.1 μM to about 10 μM, preferably from about 0.5 μM to about 5 μM, even more preferably from about 1 μM to about 3 μM. As used herein, “Endo IWR1” denotes a small molecule with chemical name [(3aR*,4S*,7R*,7aS)-1,3,3a,4,7,7a-Hexahydro-1,3-dioxo-4,7-methano-2H-isoindol-2-yl]-N-8-quinolinylbenzamide and CAS no: 1127442-82-3.

A further aspect of the present invention relates to an in vitro cell population of eye field progenitor cells, wherein at least 10%, at least 20%, at least 30%, at least 40%, at least 50%, at least 60%, at least 70%, at least 80%, or at least 90% of the eye field progenitor cells co-express PAX6 and OTX2, and one or more of VSX2 and MITF. It follows that in a particular embodiment the in vitro cell population of eye field progenitor cells is obtained by the method according to the first aspects of the present invention. By “in vitro cell population” is meant a cell population outside the human body, e.g. contained in a suitable vessel. In a particular embodiment, the eye field progenitor cells are non-native. The term “non-native” is to be understood as cells which do not occur in nature, such as in the human or animal body. Even though it is an object of stem cell therapy in general to arrive at cells identical to or as close to identical to cells in the human body then the current methods for differentiating and maturing cells do not provide cell products which are completely identical to these.

In one embodiment, the eye field progenitor cells are differentiated into neural retina progenitor cells. Accordingly, another aspect of the present invention relates to a method for obtaining neural retina progenitor cells from hPSCs. The method according to this aspect is directly related to the protocol for obtaining eye field progenitor cells. Accordingly, the embodiments relating to the method for obtaining eye field progenitor cells may equally apply to this aspect. An embodiment of this aspect relates to a method for obtaining neural retina progenitor cells, comprising the steps of culturing hPSCs, seeding the hPSCs on a substrate coated with a matrix, culturing the hPSCs in a cell culture medium to obtain differentiating cells, contacting the differentiating cells with an inhibitor of SMAD protein signaling, and contacting the differentiating cells with BMPS, wherein the differentiating cells are allowed to differentiate into neural retina progenitor cells.

In another embodiment, the eye field progenitor cells are differentiated into RPE progenitor cells.

Another aspect of the present invention therefore also relates to a method for obtaining RPE cells. The method according to this aspect is directly related to the protocol for obtaining eye field progenitor cells. Accordingly, the embodiments relating to the method for obtaining eye field progenitor cells may equally apply to this aspect. An embodiment of this aspect relates to a method for obtaining RPE progenitor cells from hPSCs, comprising the steps of culturing the hPSCs to obtain differentiating cells, contacting the differentiating cells with BMPS, contacting the differentiating cells with an inhibitor of GSK3, wherein the differentiating cells are allowed to differentiate into RPE progenitor cells. In a more specific embodiment the method for obtaining RPE progenitor cells from hPSCs, comprises the steps of culturing the hPSCs, seeding the hPSCs on a substrate coated with a matrix, culturing the hPSCs in a cell culture medium to obtain differentiating cells, contacting the differentiating cells with an inhibitor of SMAD protein signaling, contacting the differentiating cells with BMPS, and contacting the differentiating cells with an inhibitor of GSK3, wherein the differentiating cells are allowed to differentiate into RPE progenitor cells.

### GSK3

As used herein “GSK3” means Glycogen Synthase Kinase 3. GSK3 is a serine threonine kinase that takes part in many signaling pathways that, control cellular functions such as proliferation and cell polarity of neural progenitors during embryonic brain development. GSK3 acts as a downstream regulatory switch for numerous signaling pathways, including cellular responses to WNT, growth factors, insulin, receptor tyrosine kinases (RTK), Hedgehog pathways, and G-protein-coupled receptors (GPCR). Non-limiting examples of GSK3 inhibitors are CHIR99021 or CHIR, SB216763, SB415286, CHIR98014, ARA014418, 1-Azakenpaullone and Bis-7-indolylmaleimide.

As used herein “CHIR99021” and “CT99021” may be used interchangeably and refer to 6-[[2-[[4-(2,4-Dichlorophenyl)-5-(5-methyl-1H-imidazol-2-yl)-2-pyrimidinyl]amino]ethyl]amino]-3-pyridinecarbonitrile with CAS no. 252917-06-9.

In one embodiment, the inhibitor of GSK3 is CHIR99021.

In one embodiment, the differentiating cells are contacted with the inhibitor of GSK3 from about day 5 to about day 40, preferably from about day 5 to about day 25, more preferably from about day 6 to about day 26, more preferably from about day 6 to about day 25, more preferably from about day 6 to about day 24, more preferably from about day 6 to about day 23, more preferably from about day 6 to about day 22, more preferably from about day 6 to about day 21, even more preferably from about day 7 to about day 21.

In one embodiment, the differentiating cells are contacted with the inhibitor of GSK3 from about 2 days, preferably 3 days, more preferably 4 days, even more preferably 5 days after contacting the differentiating cells with BMPS or an analog thereof.

In one embodiment, the differentiating cells are contacted with the inhibitor of GSK3 in a concentration from at about 0.25 μM to about 5 μM, preferably from about 1 μM to about 4 μM, more preferably from about 2 μM to about 3 μM.

The inventors identified the inhibitor of GSK3 provides an effective and robust initiation of the differentiation towards RPE cells. The small molecule CHIR99021 furthermore facilitates translation into GMP compliance.

The present inventors contemplate that as an alternative to using a GSK3 inhibitor a WNT ligand may be used, such as WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, and WNT16. In one embodiment the WNT ligand is WNT3A.

As used herein, WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, and WNT16 are comprised in a diverse family of secreted lipid-modified signalling glycoproteins that are 350-400 amino acids in length. The type of lipid modification that occurs on these proteins is Palmitoleoylation of serine in a conserved pattern of serin residues. Palmitoleoylation is necessary because it initiates targeting of the Wnt protein to the plasma membrane for secretion and it allows the Wnt protein to bind its receptor due to the covalent attachment of fatty acids. Wnt proteins also undergo glycosylation, which attaches a carbohydrate in order to ensure proper secretion. In Wnt signaling, these proteins act as ligands to activate the different Wnt pathways via paracrine and autocrine routes.

Another aspect of the present invention relates to an in vitro cell population of eye field progenitor cells, obtained by a method according to the first embodiments of the present invention.

In one embodiment, the eye field progenitor cells of the present invention are non-native.

In one embodiment, the eye field progenitor cells including RPE progenitor cells, optic cup progenitor cells, corneal progenitor cells and NR progenitor cells are non-native.

Another aspect of the present invention relates to the use of the in vitro cell population of eye field progenitor cells for obtaining NR progenitor cells, early eye progenitor cells, and/or RPE cells.

Another aspect of the present invention relates to an in vitro cell population of RPE progenitor cells, wherein at least 10%, at least 20%, at least 30%, at least 40%, at least 50%, at least 60%, at least 70%, at least 80%, or at least 90% of the RPE progenitor cells co-express PAX6, OTX2 and MITF.

Another aspect of the present invention relates to an in vitro cell population of neural retina progenitor cells, wherein at least 10%, at least 20%, at least 30%, at least 40%, at least 50%, at least 60%, at least 70%, at least 80%, or at least 90% of the neural retina progenitor cells co-express PAX6, OTX2, and VSX2.

### Particular Embodiments

The aspects of the present invention are now further described by the following non-limiting embodiments:
1. A method for obtaining eye field progenitor cells from hPSCs, comprising the steps of:
   culturing the hPSCs to obtain differentiating cells, and
   contacting the differentiating cells with BMPS,
   wherein the differentiating cells are allowed to differentiate into eye field progenitor cells.
2. A method for obtaining eye field progenitor cells from hPSCs, comprising the steps of:
   culturing the hPSCs,
   culturing the hPSCs in a cell culture medium to obtain differentiating cells, and
   contacting the differentiating cells with BMP5,
   wherein the differentiating cells are allowed to differentiate into eye field progenitor cells.
3. A method for obtaining eye field progenitor cells from hPSCs, comprising the steps of:
   culturing the hPSCs,
   culturing the hPSCs in a cell culture medium to obtain differentiating cells,
   contacting the differentiating cells with an inhibitor of SMAD protein signaling, and
   contacting the differentiating cells with BMP5,
   wherein the differentiating cells are allowed to differentiate into eye field progenitor cells.
4. A method for obtaining eye field progenitor cells from hPSCs, comprising the steps of:
   culturing the hPSCs,
   seeding the hPSCs on a substrate coated with a matrix,
   culturing the hPSCs in a cell culture medium to obtain differentiating cells,
   contacting the differentiating cells with an inhibitor of SMAD protein signaling, and
   contacting the differentiating cells with BMP5,
   wherein the differentiating cells are allowed to differentiate into eye field progenitor cells.
5. The method according to the preceding embodiment, wherein the differentiating cells are contacted with BMP5 or an analog thereof, wherein the analog thereof is an effective activator of the bone morphogenetic protein (BMP) signaling pathway.
6. The method according to any one of the preceding embodiments, wherein the differentiating cells are contacted with BMP5 (SEQ ID NO: 1), wherein the analog has at least 50%, 60% 70%, 80%, 90%, 91%, 92%, 93%, 94%, 95%, 96%, 97%, 98%, or 99% identity with BMP5 identified by SEQ ID NO: 1, wherein the analog is an effective activator of the bone morphogenetic protein (BMP) signaling pathway.
7. The method according to any one of the preceding embodiments, wherein the differentiating cells are contacted with an effective amount of BMP5 or an analog thereof.
8. The method according to any one of the preceding embodiments, wherein the differentiating cells are contacted with BMP5 (SEQ ID NO: 1).
9. The method according to any one of the preceding embodiments, wherein the concentration of BMP5 is at least about 10, 20, 30, 40, 50, 60, 70, 80, 90, 100, 110, 120, 130, 140, 150, 160, 170, 180, 190, or 200 ng/ml, preferably at least about 150 ng/ml, more preferably at least about 180 ng/ml.
10. The method according to any one of the preceding embodiments, wherein the concentration of BMP5 is below about 1000, 900, 800, 700, 600, or 500 ng/ml, preferably below about 450 ng/ml.
11. The method according to any one of the preceding embodiments, wherein the concentration of BMP5 is from about 100 ng/ml to about 600 ng/ml, preferably from about 150 ng/ml to about 550 ng/ml, more preferably from about 200 ng/ml to about 500 ng/ml, more preferably from about 200 ng/ml to about 400 ng/ml.
12. The method according to any one of the preceding embodiments, wherein the concentration of BMP5 is from about 350 ng/ml to about 450 ng/ml, preferably from about 360 ng/ml to about 440 ng/ml, more preferably from about 370 ng/ml to about 430 ng/ml, more preferably from about 380 ng/ml to about 420 ng/ml, more preferably from about 390 ng/ml to about 410 ng/ml, even more preferably about 400 ng/ml.
13. The method according to any one of the preceding embodiments, wherein the activity (ED₅₀) of BMP5 is from about 0.1 μg/ml to about 2 μg/ml, preferably from about 0.15 μg/ml to about 1.5 μg/ml, more preferably from about 0.2 μg/ml to about 1.3 μg/ml, even more preferably from about 0.21 μg/ml to about 1.2 μg/ml.
14. The method according to any one of the preceding embodiments, wherein the differentiating cells are contacted with BMP5 from at about day 5 to at about day 14, preferably from at about day 6 to at about day 13, more preferably from at about day 6 to at about day 12, more preferably from about day 6 to about day 11, more preferably from about day 6 to about day 10, more preferably from about day 6 to about day 9, more preferably from about day 6 to about day 8, even more preferably from about day 7.
15. The method according to any one of the preceding embodiments, wherein the differentiating cells are contacted BMP5 or an analog thereof, and wherein the differentiating cells are allowed to differentiate into differentiate into eye field progenitor cells until about day 30, 29, 28, 27, 26, 25, 24, 23, 22, 21, 20, or 19, preferably until about day 20 to day 22, even more preferably until about day 21.
16. The method according to any one of the preceding embodiments, wherein the differentiating cells are contacted with BMP5 from about day 5 to about day 30, from about day 5 to about day 29, from about day 5 to about day 28, from about day 5 to about day 27, preferably from about day 6 to about day 26, more preferably from about day 6 to about day 25, more preferably from about day 6 to about day 24, more preferably from about day 6 to about day 23, more preferably from about day 6 to about day 22, more preferably from about day 6 to about day 21, even more preferably from about day 7 to about day 21.
17. The method according to any one of the preceding embodiments, wherein the differentiating cells are allowed to differentiate into eye field progenitor cells for about 15 to 40 days, preferably about 17 days to 25 days, preferably for about 18 days to 24 days, more preferably for about 19 days to 23 days, more preferably for about 19 days to 23 days, more preferably for about 20 days to 22 days, even more preferably for about 21 days, starting from day 0.
18. The method according to any one of the preceding embodiments, further comprising the step of:
   contacting the differentiating cells with an inhibitor of the WNT/β-catenin pathway.
19. The method according to embodiment 18, wherein the cells are contacted with the inhibitor of the WNT/β-catenin pathway from about day 2 to about day 15, preferably from about day 3 to about day 14, more preferably from about day 3 to about day 13, even more preferably from about day 3 to about day 12.
20. The method according to any one of the embodiments 18 and 19, wherein the inhibitor of the WNT/β-catenin pathway is Endo IWR 1.
21. The method according to embodiment 20, wherein the concentration of Endo IWR 1 is from about 0.1 μM to about 10 μM, preferably from about 0.5 μM to about 5 μM, even more preferably from about 1 μM to about 3 μM.
22. The method according to any one of the embodiments 4 to 21, wherein the matrix comprises a laminin or a fragment thereof.
23. The method according to embodiment 22, wherein the laminin or a fragment thereof selected from the group consisting of laminin-511 and laminin-332.
24. The method according to embodiment 22, wherein the laminin or fragment thereof is a combination of laminin-511 and laminin-332.
25. The method according to embodiment 23, wherein the laminin or fragment thereof is laminin-332.
26. The method according to any one of the embodiments 22 to 25, wherein the laminin is an intact laminin protein.
27. The method according to any one of the embodiments 22 to 25, wherein the laminin is a fragment of the intact laminin protein.
28. The method according to any one of the embodiments 22 to 27, wherein the concentration of the laminin is from about 0.01 μg/cm² to about 50 μg/cm², preferably from about 0.1 μg/cm² to about 25 μg/cm², more preferably from about 0.1 μg/cm² to 10 μg/cm2, more preferably from about 0.1 μg/cm² to about 5, more preferably from about 0.25 μg/cm² to about 1 μg/cm², even more preferably about 0.5 μg/cm².
29. The method according to any one of the embodiments 3 to 28, wherein the differentiating cells are contacted with an inhibitor of SMAD protein signaling selected from the group consisting of GW788388, LDN-193189, LY2157299, LY364947, NOGGIN, RepSOX, SB431542, and TEW-7197.
30. The method according to embodiment 29, wherein the differentiating cells are contacted with are contacted with a combination of inhibitors of SMAD protein signaling, wherein at least one of the inhibitors of SMAD protein signaling selected from the group consisting of GW788388, LDN-193189, LY2157299, LY364947, NOGGIN, RepSOX, SB431542, and TEW-7197.
31. The method according to any one of the embodiments 3 to 30, wherein the differentiating cells are contacted with an inhibitor of SMAD protein signaling selected from the group consisting of GW788388 and/or RepSOX.
32. The method according to any one of embodiments 29 and 31, wherein the inhibitor of SMAD protein signaling is GW788388.
33. The method according to embodiment 32, wherein the inhibitor of SMAD protein signaling is GW788388 in a concentration of from 0.1 ng/ml to 1000 ng/ml, preferably from about 5 ng/ml to about 1000 ng/ml, more preferably from about 10 ng/ml to about 500 ng/ml.
34. The method according to any one of embodiments 29 and 31, wherein the inhibitor of SMAD protein signaling is RepSOX.
35. The method according to embodiment 34, wherein the inhibitor of SMAD protein signaling is RepSOX in a concentration of from 0.25 μM to 200 μM, preferably from about 10 μM to about 150 μM, more preferably from about 15 μM to about 100 μM, even more preferably from about 20 μM to about 75 μM.
36. The method according to any one of the embodiments 3 to 35, wherein the differentiating cells are contacted with only one inhibitor of SMAD protein signaling.
37. The method according to any one of the embodiments 3 to 36, wherein the differentiating cells are contacted with the SMAD protein signaling from about day 0.
38. The method according to any one of the preceding embodiments, wherein the differentiating cells are contacted BMP5 or an analog thereof, wherein the differentiating cells are allowed to differentiate into differentiate into eye field progenitor cells until about day 40 or longer, day 39, 38, 37, 36, 35, 34, 33, 32, 31, 30, 29, 28, 27, 26, 25, 24, 23, 22, 21, 20, or 19, preferably until about day 20 to day 22, even more preferably until about day 21.
39. The method according to any one of the embodiments 3 to 38, wherein the differentiating cells are contacted with the inhibitor of SMAD protein signaling from about day 0 to about day 15, preferably to about day 14, more preferably to about day 13, and even more preferably to about day 12.
40. The method according to any one of the embodiments 3 to 39, wherein the differentiating cells are contacted with an inhibitor of SMAD protein signaling from about day 0 to about day 12, and the differentiating cells are contacted with BMP5from about day 7, wherein the differentiating cells are allowed to differentiate into eye field progenitor cells until about day 21.
41. The method according to any one of the preceding embodiments, wherein at least 10%, at least 20%, at least 30%, at least 40%, at least 50%, at least 60%, at least 70%, at least 80%, or at least 90% of the eye field progenitor cells co-express PAX6 and OTX2, and one or more of VSX2 and MITF.
42. The method according to any one of the preceding embodiments, wherein at least 50%, at least 60%, at least 70%, at least 80%, or at least 90% of the eye field progenitor cells co-express PAX6 and OTX2, and at least 10%, at least 20%, at least 30%, at least 40%, at least 50% of the eye field progenitor cells further co-express one or more of VSX2 and MITF.
43. The method according to any one of the embodiments 2 to 42, wherein the cell culture medium is chemically defined and xeno-free.
44. The method according to any one of the embodiments 2 to 43, wherein the cell culture medium is feeder cell-free.
45. The method according to any one of the embodiments 2 to 44, wherein the hPSCs are plated with a density of from about 10,000 cells per cm² to about 100,000 cells per cm², preferably from about 20,000 cells per cm² to about 80,000 cells per cm², more preferably from about 30,000 cells per cm² to about 50,000 cells per cm², even more preferred about 40,000 cells per cm².
46. The method according to any one of the embodiments 2 to 45, wherein the cell culture medium at day 0 is a first cell culture medium and wherein at least part of the cell culture medium is replaced with a second cell culture medium from about day 1.
47. The method according to any one of the embodiments 2 to 46, wherein the cell culture medium at day 0 is a first cell culture medium and wherein the first cell culture medium is substantially replaced with a second cell culture medium from about day 1.
48. The method according to any one of embodiments 46 and 47, wherein the first cell culture medium is Nutristem®, such as Nutristem® hPSC XF Medium for iPS and ES Stem Cells.
49. The method according to any one of the embodiments 46 to 48, wherein the first cell culture medium further comprises a ROCK inhibitor, preferably the ROCK inhibitor is Y-27632.
50. The method according to any one of the embodiments 46 to 49, wherein the second cell culture medium comprises GM EM or DMEM/F12 supplemented with N2 and B27.
51. The method according to any one of the preceding embodiments, wherein the eye field progenitor cells are further differentiated into NR cells.
52. The method according to any one of the preceding embodiments, wherein the eye field progenitor cells are differentiated into RPE cells.
53. The method according to any one of the preceding embodiments, wherein the eye field progenitor cells are RPE progenitor cells, and wherein said method further comprises the step of contacting the differentiating cells with an inhibitor of GSK3.
54. The method according to embodiment 53, wherein the inhibitor of GSK3 is CHIR99021.
55. The method according to embodiment 54, wherein the concentration of CHI R99021 is from at about 0.25 μM to about 5 μM, preferably from about 1 μM to about 4 μM, more preferably from about 2 μM to about 3 μM.
56. The method according to any one for the embodiments 53 to 55, wherein the differentiating cells are contacted with the inhibitor of GSK3 from about day 5 to about day 40, preferably from about day 5 to about day 25, more preferably from about day 6 to about day 26, more preferably from about day 6 to about day 25, more preferably from about day 6 to about day 24, more preferably from about day 6 to about day 23, more preferably from about day 6 to about day 22, more preferably from about day 6 to about day 21, even more preferably from about day 7 to about day 21.
57. The method according to any one for the embodiments 53 to 56, wherein the differentiating cells are contacted with the inhibitor of GSK3 from at about day 7 to at about day 15, preferably from at about day 12.
58. The method according to any one of the embodiments 53 to 57, wherein the differentiating cells are contacted with the inhibitor of GSK3 from about 2 days, preferably 3 days, more preferably 4 days, even more preferably 5 days after contacting the differentiating cells with BMPS.
59. An in vitro cell population of eye field progenitor cells, wherein at least 10%, at least 20%, at least 30%, at least 40%, at least 50%, at least 60%, at least 70%, at least 80%, or at least 90% of the eye field progenitor cells co-express PAX6 and OTX2, and one or more of VSX2 and MITF.
60. The in vitro cell population of eye field progenitor cells according to embodiment 59, wherein the eye field progenitor cells are non-native.
61. The in vitro cell population according to any one of embodiments 59 and 60, wherein the eye field progenitor cells are RPE progenitor cells.
62. The in vitro cell population according to any one of embodiments 59 and 60, wherein the eye field progenitor cells are NR progenitor cells.
63. An in vitro cell population of eye field progenitor cells, obtained by the method according any one of the embodiments 1 to 58.
64. Use of the in vitro cell population of eye field progenitor cells according any one of the embodiments 59 to 63 for obtaining NR progenitor cells, early eye progenitor cells, and/or RPE progenitor cells.
65. Use according to embodiment 64 for the treatment of an eye condition, such as age-related macular degeneration, cataracts, cornea blindness, glaucoma and RP.
66. A method for obtaining RPE progenitor cells from hPSCs, comprising the steps of:
   culturing the hPSCs,
   seeding the hPSCs on a substrate coated with a matrix,
   culturing the hPSCs in a cell culture medium to obtain differentiating cells,
   contacting the differentiating cells with an inhibitor of SMAD protein signaling,
   contacting the differentiating cells with BM P5 or an analog thereof, and
   contacting the differentiating cells with an inhibitor of GSK3,
   wherein the differentiating cells are allowed to differentiate into RPE progenitor cells.
67. An in vitro cell population of RPE progenitor cells, wherein at least 10%, at least 20%, at least 30%, at least 40%, at least 50%, at least 60%, at least 70%, at least 80%, or at least 90% of the RPE progenitor cells co-express PAX6, OTX2, and MITF.
68. The in vitro cell population of RPE progenitor cells according to embodiment 67, wherein the RPE progenitor cells are non-native.
69. A method for obtaining neural retina progenitor cells from hPSCs, comprising the steps of:
   culturing the hPSCs,
   seeding the hPSCs on a substrate coated with a matrix,
   culturing the hPSCs in a cell culture medium to obtain differentiating cells,
   contacting the differentiating cells with an inhibitor of SMAD protein signaling, and
   contacting the differentiating cells with BMP5,
   wherein the differentiating cells are allowed to differentiate into neural retina progenitor cells.
70. An in vitro cell population of neural retina progenitor cells, wherein at least 10%, at least 20%, at least 30%, at least 40%, at least 50%, at least 60%, at least 70%, at least 80%, or at least 90% of the neural retina progenitor cells co-express PAX6, OTX2, and VSX2.
71. The in vitro cell population of neural retina progenitor cells according to embodiment 70, wherein the neural retina progenitor cells are non-native.

### EXAMPLES

The following are non-limiting examples of protocols for carrying out the invention.

General Methods of Preparation

Culture of hESCs

An internally generated hESC line was maintained on human recombinant laminin (hrLN) coated plates (Biolaminin 521 LN, Biolamina) in NutriStem hPSC XF medium (Biological Industries), in a 5% CO₂ incubator at 37° C. and passaged enzymatically at 1:10-1:20 ratio every 3-5 days. For passaging, confluent cultures were washed once with phosphate buffered saline (PBS) without calcium and magnesium ions and incubated for 5 min at 37° C. with TrypLE Select (GIBCO, Thermo Fisher Scientific). The enzyme was then carefully removed and the cells were collected in fresh NutriStem hPSC XF medium by gentle pipetting to obtain single cell suspension and the required volume plated on a freshly hrLN-521 coated dish. After passage, the medium was replaced with fresh prewarmed NutriStem hPSC XF medium and changed daily.

Differentiation of hESCs into Eye Field Progenitor Cells

hESC-RPE monolayer differentiation hESC were plated at a cell density of 5.5×10⁴ cells/cm2 on hrLN-332 laminin coated dishes at 10 μg/mL (Biolaminin 332 LN, Biolamina) using NutriStem hPSC XF medium. Rho-kinase inhibitor (Y-27632, Millipore) at a concentration of 10 μM was added during the first 24 hours, while cells were kept at 37° C., 5% CO₂. After 24 hours, the culture medium was replaced with differentiation medium according to the examples. The differentiation media “GMEM” in the following examples is always supplemented with Penicillin-Streptomycin solution (20 units/ml; Thermo Fisher), beta-Mercaptoethanol (0.5 mM; Thermo Fisher), Sodium pyruvate (1 mM; Thermo Fisher), Non-Essential Amino Acids (1×; Thermo Fisher).

### Concentrations

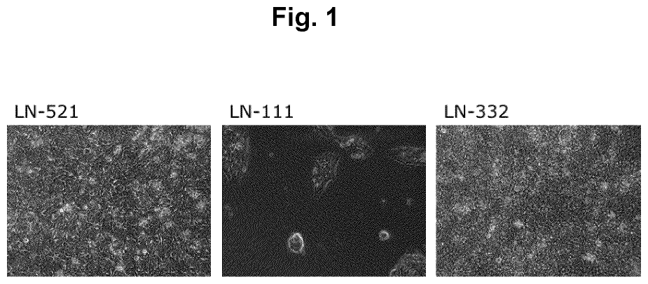

The following concentrations presented in table 1 may be used in the protocols provided in examples 1 to 11. These concentrations were also used in the experiments carried out and referred to in FIGS. 1 to 15.

TABLE 1

| Name | Concentration | | Compound | Vendor |
|---|---|---|---|---|
| NOGGIN | 100 | ng/ml | Recombinant protein | Peprotech |
| elWR1 | 1 | μM | Small Molecule | Tocris |
| BMP5 | 200 | ng/ml | Recombinant protein | R&D systems |
| CHIR99021 | 2 | μM | Small molecule | StemMACS |
| IHH | 200 | ng/ml | Recombinant protein | R&D systems |
| GW788388 | 10 | ng/ml | Small molecule | Tocris |
| RepSOX | 25 | μM | Small molecule | Tocris |
| ROCKi (Y-27632) | 10 | μM | Small molecule | StemMACS |
| DKK2 | 100 | ng/ml | Recombinant protein | R&D systems |
| Activin A | 100 | ng/ml | Recombinant protein | R&D systems/Peprotech |

| | | | |
|---|---|---|---|
| B-27 w/o vitamin A | 2% | Supplement | Gibco |
| (50x) | | | |
| N-2 (100X) | 1% | Supplement | Gibco |

### Example 1

Protocol for Obtaining Eye Field Progenitor Cells Using Dual SMAD Inhibition and WNT Inhibition in Combination with BMP5

In order to differentiate hESC into eye progenitor cells, we tested the effect of BMP5 in different conditions in combination with small molecules and recombinant proteins that mimics developmental cues. We relied on sequential activation and/or repression of the common developmental pathways associated to eye development for our differentiation protocol and combined with activation of BMP pathway by BMP5. After cell dissociation, hESC line expanded on LN-521 were dissociated to single cells and seeded on LN-332 for differentiation. These cells adapted well to this laminin. A comparison in cell adhesion after 12 hours between LN-521, LN-111 and LN-332 is shown in FIG. 1.

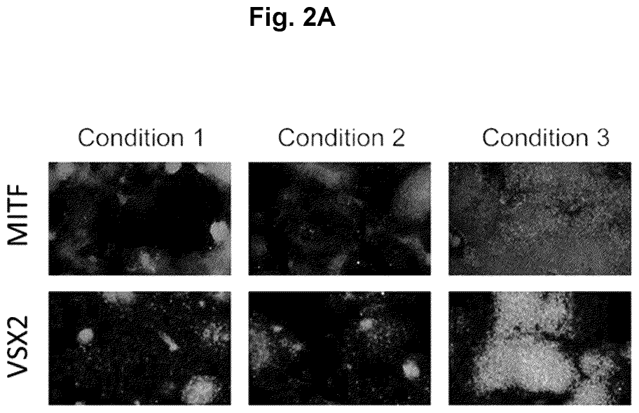

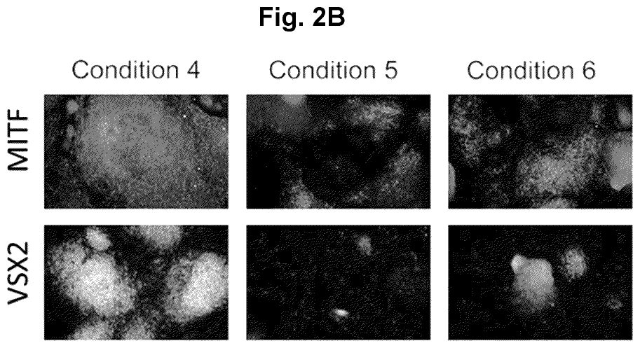

Here we summarize the different 6 conditions tested for differentiation shown in FIG. 2.

Condition 1: Control condition using a dual SMAD inhibition, WNT inhibition, without BMP5

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-11: 100% (GMEM+1% (v/v) N2+2% (v/v) B27)+NOGGIN+GW788388+Endo IWR1

Day 12-21: 100% (GMEM+1% (v/v) N2+2% (v/v) B27)

Condition 2: Condition Using a Dual SMAD Inhibition, Sequential WNT Inhibition, without BMP5

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-11: 100% (GMEM+1% (v/v) N2+2% (v/v) B27)+NOGGIN+GW788388+Endo IWR1

Day 12-21: 100% (GMEM+1% (v/v) N2+2% (v/v) B27)+IHH+DKK2

Condition 3: Condition Using a Dual SMAD Inhibition, Sequential WNT Inhibition, with BMP5

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-11: 100% (GMEM+1% (v/v) N2+2% (v/v) B27)+NOGGIN+GW788388+Endo IWR1

Day 12-21: 100% (GMEM+1% (v/v) N2+2% (v/v) B27)+IHH+DKK2+BMP5

Condition 4: Condition Using a Dual SMAD Inhibition, Sequential WNT Inhibition, with BMP5 and Activin A

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-11: 100% (GMEM+1% (v/v) N2+2% (v/v) B27)+NOGGIN+GW788388+Endo IWR1

Day 12-14: 100% (GMEM+1% (v/v) N2+2% (v/v) B27)+IHH+DKK2+BMP5

Day 15-21: 100% (GMEM+1% (v/v) N2+2% (v/v) B27)+IHH+DKK2+BMP5+Activin A

Condition 5: Condition Using a Dual SMAD Inhibition, Sequential WNT Inhibition, with Activin A, without BMP5

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-11: 100% (GMEM+1% (v/v) N2+2% (v/v) B27)+NOGGIN+GW788388+Endo IWR1

Day 12-21: 100% (GMEM+1% (v/v) N2+2% (v/v) B27)+IHH+DKK2+Activin A

Condition 6: Control Condition Using a Dual SMAD Inhibition, Sequential WNT Inhibition, without BMP5

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-11: 100% (GMEM+1% (v/v) N2+2% (v/v) B27)+NOGGIN+GW788388+Endo IWR1

Day 12-14: 100% (GMEM+1% (v/v) N2+2% (v/v) B27)+IHH+DKK2

Day 15-21: 100% (GMEM+1% (v/v) N2+2% (v/v) B27)+IHH+DKK2+Activin A

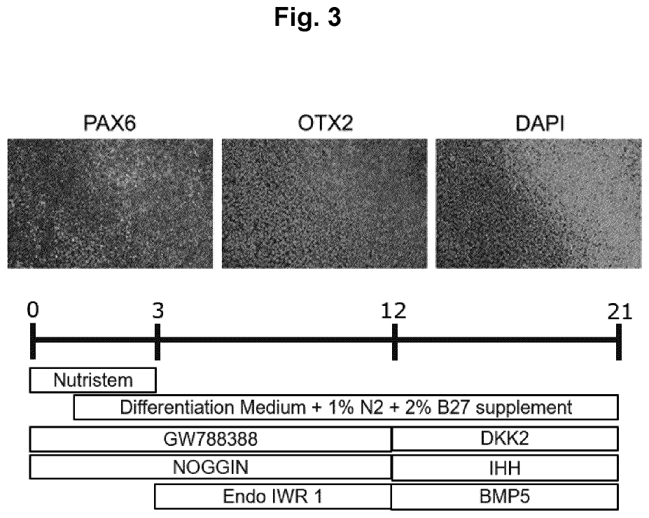

As show in FIGS. 2A and B, the use of BMP5 in condition 3 generated MITF and VSX2-positive cells. Stimulation of Hedgehog pathway by IHH and the use of the WNT inhibitor DKK2, was not sufficient to generate MITF or VSX2-positive cells, as shown in condition 2, that shows a similar pattern as control (condition 1). The treatment with Activin A together with BMP5 did not increased the levels of MITF or VSX2, as shown in condition 4. In contrast, the replacement of BMP5 by Activin A or the treatment with BMP5 after an initial treatment with Activin A had a negative effect in the generation of MITF and VSX2-positive cells, as shown in conditions 5 and 6. As shown in FIG. 3, these cells show forebrain identity, positive for PAX6 and OTX2 markers, compatible with eye field progenitor cell identity.

These results indicate that BMP5 strongly generates eye field progenitor cells positive for the RPE progenitor cell marker MITF and the NR progenitor cell marker VSX2. The addition of IHH, DKK2 and Activin A did not show any additive effect to the treatment with BMP5 on expression of these markers (FIGS. 2A and B).

### Example 2

Protocol for Obtaining Eye Field Progenitor Cells Using Dual SMAD Inhibition in Combination with BMP5 and CHIR99021

We decided to explore the inhibition of GSK3 with the small molecule CHIR99021 in combination with BMP5, and to remove the WNT inhibitor Endo IWR1 from the protocol, as GSK3 inhibition might activate canonical (beta-Catenin dependent) WNT signalling. As BMP5 showed a positive effect on the generation of MITF and VSX2-positive cells, we tested an earlier time point for BMP5 treatment, starting at day 7.

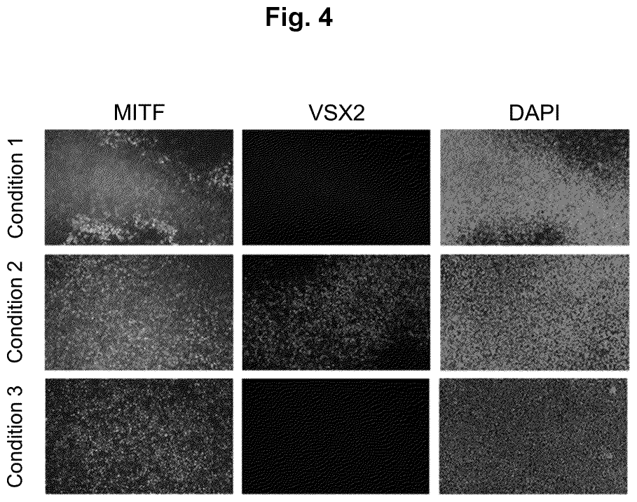

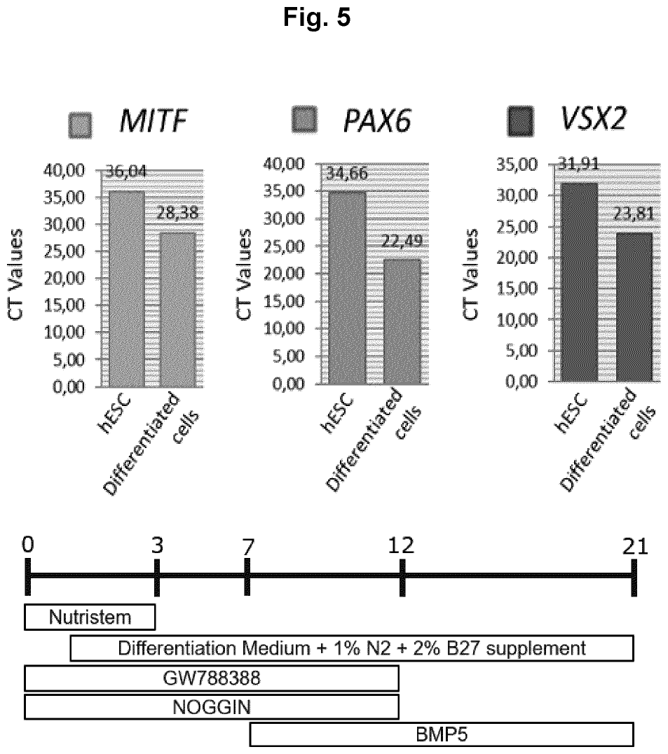

Here we summarize the different 3 conditions tested and shown in FIG. 4, and complemented with FIGS. 5, 6 and 7.

Condition 2: Control Condition Using a Dual SMAD Inhibition, without BMP5

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-11: 100% (GMEM+1% (v/v) N2+2% (v/v) B27)+NOGGIN+GW788388

Day 12-21: 100% (GMEM+1% (v/v) N2+2% (v/v) B27)

Condition 2: Condition Using a Dual SMAD Inhibition, with BMP5

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-6: 100% (GMEM+1% (v/v) N2+2% (v/v) B27)+NOGGIN+GW788388

Day 7-11: 100% (GMEM+1% (v/v) N2+2% (v/v) B27)+NOGGIN+GW788388+BMP5

Day 12-21: 100% (GMEM+1% (v/v) N2+2% (v/v) B27)+BMP5

Condition 3: Condition Using a Dual SMAD Inhibition, with BMP5 and CHIR99021

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-6: 100% (GMEM+1% (v/v) N2+2% (v/v) B27)+NOGGIN+GW788388

Day 7-11: 100% (GMEM+1% (v/v) N2+2% (v/v) B27)+NOGGIN+GW788388+BMP5

Day 12-21: 100% (GMEM+1% (v/v) N2+2% (v/v) B27)+BMP5+CHIR99021

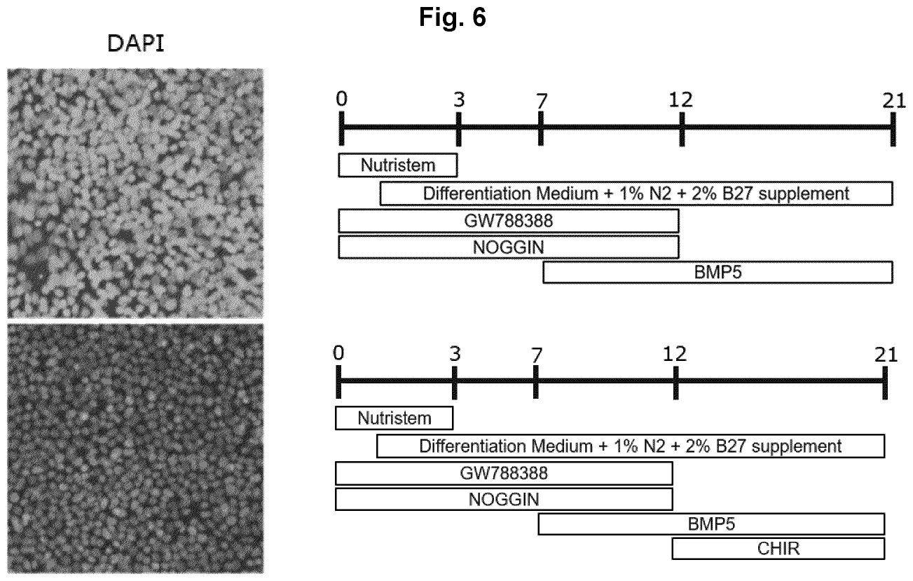

As shown in FIG. 4 condition 2, BMP5 strongly generated MITF and VSX2-positive cells. MITF, PAX6 and VSX2 mRNA levels were also increased compared to hESC, as the CT values were significantly decreased (FIG. 5). When BMP5 was combined with CHIR99021, the numbers of VSX2-positive cells were drastically reduced (condition 3, FIG. 4). Surprisingly, the MITF-positive cells under the BMP5/CHIR99021 treatment adopted a well-organized cobblestone morphology, characteristic of RPE progenitor cells, compared to the condition with only BMP5 (FIG. 6).

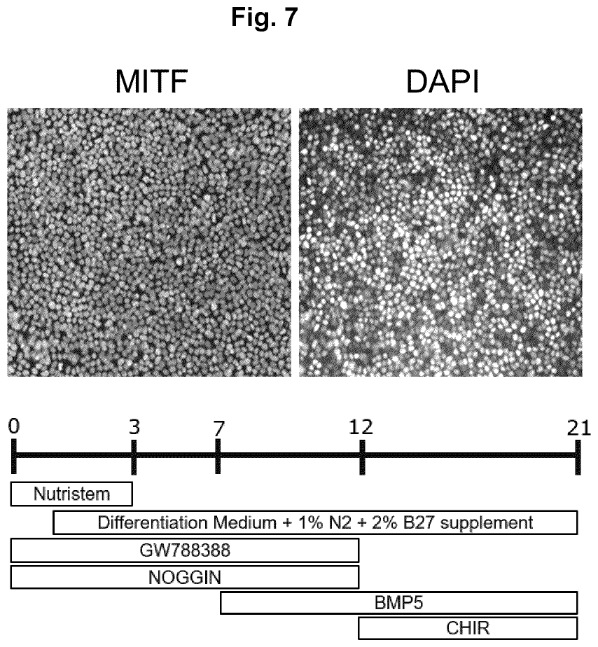

The combination of BMP5 and CHIR99021 generated a very homogeneous cell population of MITF-positive cells after 21 days, as shown in FIG. 7.

In summary, our sequential BMP5-based protocol differentiates hESCs into eye field progenitor cells (MITF/VSX2), and the addition of CHIR99021 can redirect these cells to a more RPE progenitor cell identity, positive for MITF and negative for VSX2, with a cobblestone morphology.

### Example 3

Protocol for Obtaining Eye Progenitor Cells Using RepSOX and NOGGIN as Dual SMAD Inhibitors, with BMP5 and CHIR99021

We decided to explore the effect of another SMAD inhibitor, RepSOX, in combination with NOGGIN, and to combine with BMP5 and CHIR99021 to obtain RPE progenitor cells positive for MITF. This will indicate if different SMAD inhibitors could be used in our BMP5-based protocol.

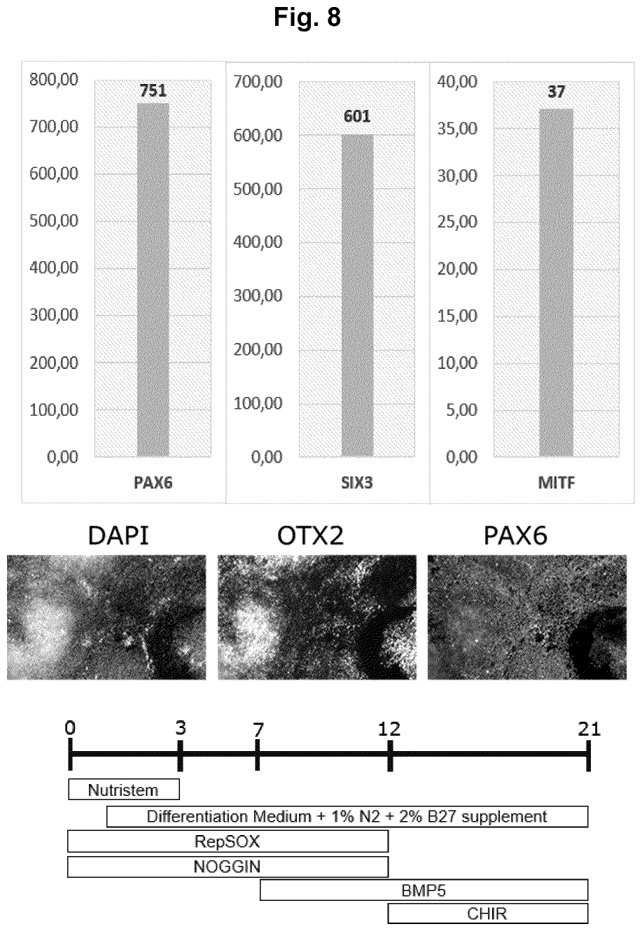

Here we summarize the condition tested as shown in FIG. 8.

Condition RepSOX: Dual SMAD Inhibition with BMP5 and CHIR99021

Day 0: 100% Nutristem+RepSOX+NOGGIN+Y-27632

Day 1: 50% Nutristem+50% (GMEM+1% (v/v) N2+2% (v/v) B27)+RepSOX+NOGGIN

Day 2: 75% Nutristem+25% (GMEM+1% (v/v) N2+2% (v/v) B27)+RepSOX+NOGGIN

Day 3-6: 100% (GMEM+1% (v/v) N2+2% (v/v) B27)+NOGGIN+GW788388

Day 7-11: 100% (GMEM+1% (v/v) N2+2% (v/v) B27)+RepSOX+NOGGIN+BMP5

Day 12-21: 100% (GMEM+1% (v/v) N2+2% (v/v) B27)+BMP5+CHIR99021

As shown in FIG. 8, there was a clear induction of PAX6, SIX3 and MITF gene expression after 21 days, indicating the generation of eye field progenitor cells with RPE progenitor cell identity. At protein level, RepSOX in combination with BMP5 and CHIR99021 also generated OTX2 and PAX6-positive cells assessed by immunostaining, markers for eye field progenitor cells.

In conclusion, these results indicate that different SMAD inhibitors can be used in our BMP5-based protocol to generate eye field progenitor cells.

### Example 4

Protocol for Obtaining Eye Field Progenitor Cells Using Single SMAD Inhibition and BMP5 or BMP5 with CHIR99021

In order to provide quantitative data of the cells generated with our BMP5-based protocol, we analysed the eye field progenitor cells generated under treatment with BMP5 for NR progenitor cells or with BMP5 and CHIR99021 for RPE progenitor cells by flow cytometry. Moreover, we tested single SMAD inhibition by removing NOGGIN from the protocol.

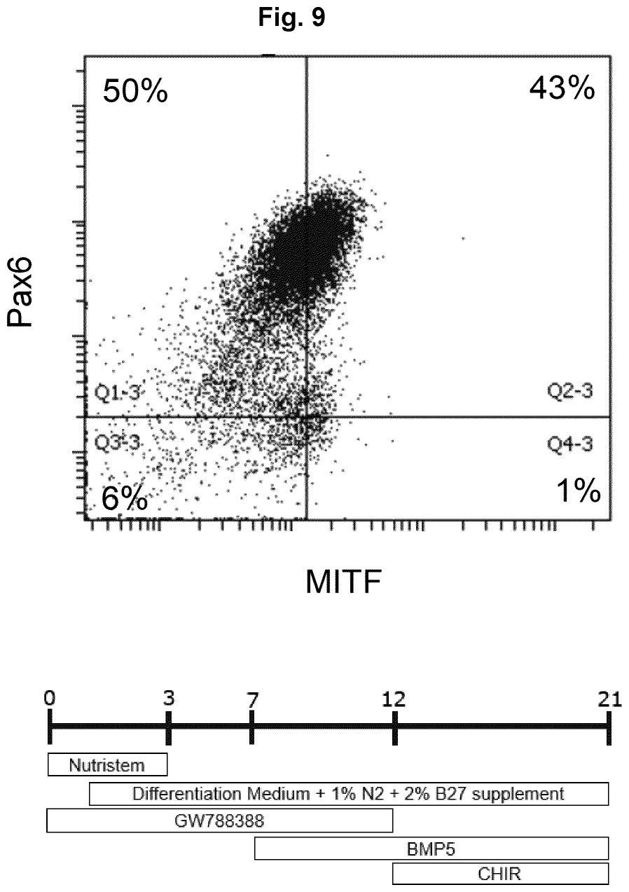

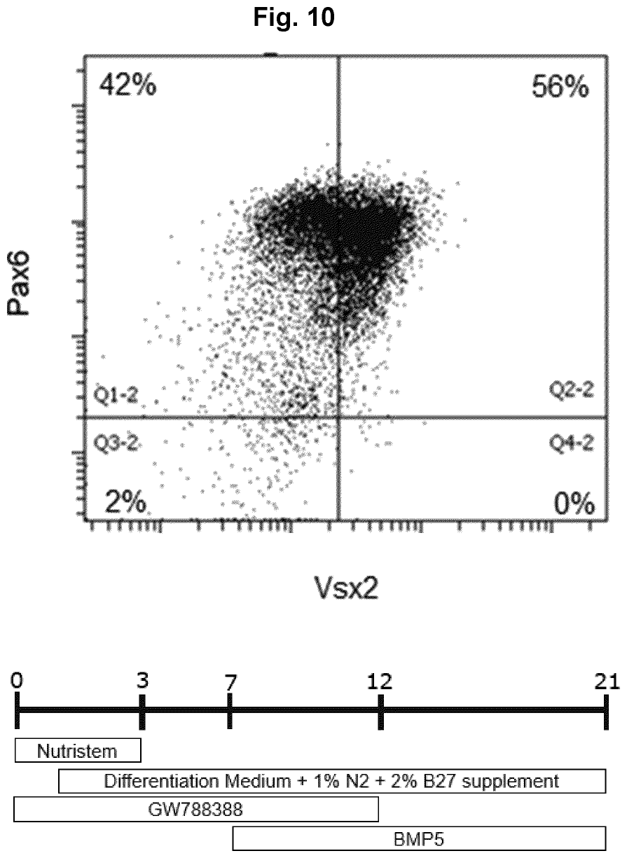

Here we summarize 2 different conditions tested and shown in FIGS. 9 and 10.

Condition for RPE (FIG. 9): Single SMAD Inhibition with BMP5 and CHIR99021

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388

Day 7-11: 100% (GMEM+1% (v/v) N2+2% (v/v) B27)+GW788388+BMP5

Day 12-21: 100% (GMEM+1% (v/v) N2+2% (v/v) B27)+BMP5+CHIR99021

Condition for NR (FIG. 10): Single SMAD Inhibition and BMP5

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388

Day 7-11: 100% (GMEM+1% (v/v) N2+2% (v/v) B27)+GW788388 +BMP5

Day 12-21: 100% (GMEM+1% (v/v) N2+2% (v/v) B27)+BMP5

As shown in FIG. 9, PAX6-positive cells represent more than 90% of the differentiated cells, with more than 40% being positive for both PAX6/MITF after 21 days when cells are exposed to BMP5 and CHIR99021. This indicates that the eye field progenitor cells adopt an RPE progenitor cell identity after being exposed to a single SMAD inhibitor (GW788388) and a subsequent treatment with BMP5 and CHIR99021. When cells were exposed to BMP5 alone, the number of PAX6 positive cells represented more than 95%, and the double positive cells PAX6/VSX2 were more than 50%, as shown in FIG. 10. This indicates that single SMAD inhibition in combination with BMP5 generated eye field progenitor cells with a NR progenitor cell identity, that are positive for PAX6 and VSX2.

In conclusion, single SMAD inhibition in our BMP5-based protocol generated more than 50% of eye field progenitor cells with a NR progenitor identity (PAX6/VSX2), and the addition of CHIR99021 can redirect these cells to a RPE progenitor cells (MITF) identity. Surprisingly, the use of a single SMAD inhibitor (GW788388) in our BMP5-based protocol can replace the use of a dual SMAD inhibition.

### Example 5

Single Cell RNA Sequencing Analysis on a Protocol for Obtaining Eye Field Progenitor Cells with a RPE Progenitor Cell Identity Using Single SMAD Inhibition with BMP5 and CHIR99021

In order to evaluate the effect of the single SMAD inhibition in our BMP5-based protocol, in combination with CHIR99021 for the generation of eye field progenitor cells with a RPE progenitor cell identity at the transcriptomic level, we performed single cell RNA sequencing (scRNAseq) at day 21 on cells that were generated with the following protocol,

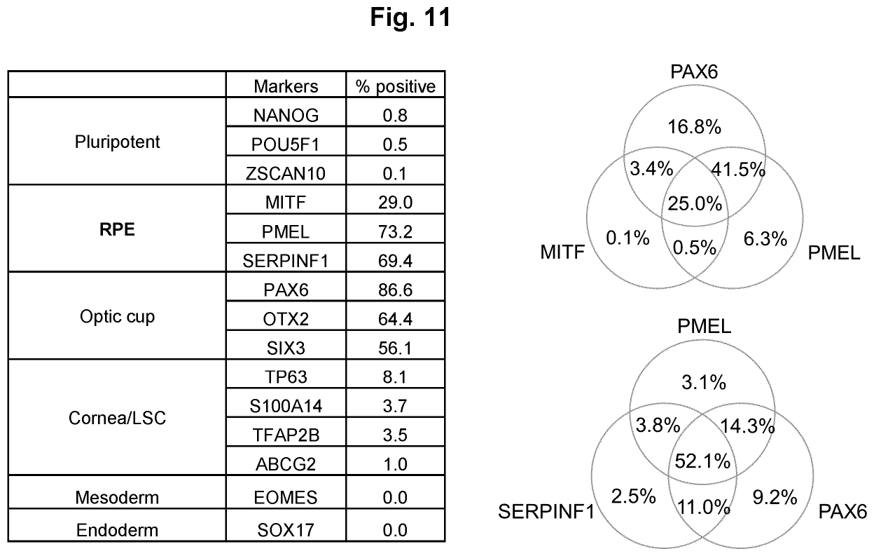

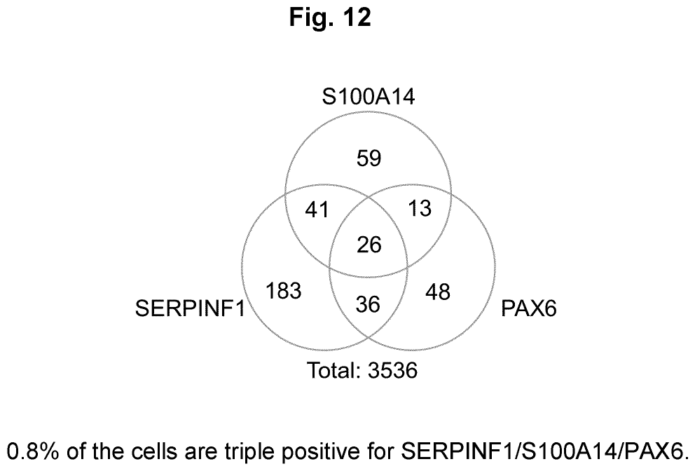

Here we summarize the tested condition for FIGS. 11 and 12.

Condition: Single SMAD inhibition and BMP5+CHIR99021 treatment:

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388

Day 7-11: 100% (GMEM+1% (v/v) N2+2% (v/v) B27)+GW788388+BMP5

Day 12-21: 100% (GMEM+1% (v/v) N2+2% (v/v) B27)+BMP5+CHIR99021

The table in FIG. 11 shows the percentages of hESC-derived eye field progenitor cells expressing the indicated genes, analysed by scRNAseq. As shown in the table, the markers for pluripotency (NANOG, POU5F1 and ZSCAN10) represented less than 1% of the cells after 21 days of differentiation, and no triple NANOG/POU5F1/ZSCAN10 positive cells were detected, indicating that pluripotent cells were not present. Cells positive for specific markers for RPE progenitor cells were detected, with MITF representing 29%, PMEL 73% and SERPINF1 69%. Cells positive for optic cup markers such as PAX6, OTX2 and SIX3 were also detected (86%, 64% and 56%, respectively). Of note, no cells positive for other germ cell lineages such as mesoderm and endoderm lineages were detected. Each Venn diagram shows expression patterns of cells co-expressing genes characteristic of RPE progenitor cells, PAX6/MITF/PMEL and PAX6/PMEL/SERPINF1 genes.

Surprisingly, we identified a small cluster of cells positive for LSC (also known as corneal stem cells) markers, such as TP63 (8%), S100A14 (4%), TFAP2B (4%) and ABCG2 (1%). In FIG. 12, we represent the Venn diagram showing triple positive cells for LSC (TP63/TFAP2B/S100A14), representing 0.8% of cells.

In conclusion, our BMP5-based protocol in combination with CHIR99021 and single SMAD inhibition generated eye field progenitor cells with a RPE progenitor cell identity (MITF/PMEL/SERPINF1). Undifferentiated cells or cells from mesodermal or endodermal linages were absent. Surprisingly, our BMP5-based protocol is also capable of generating eye field progenitor cells with a LSC (also known as corneal stem cells) identity.

### Example 6

Protocol for Obtaining Eye Field Progenitor Cells Using Different Concentrations of BMP5

In order to determine which concentration range of BMP5 is the most effective to generate eye field progenitor cells, we tested three different concentration (0.1, 200 and 1000 ng/ml) and compared to the control condition without BMP5. We analyzed gene expression at day 21 on cells that were generated with the following protocol,

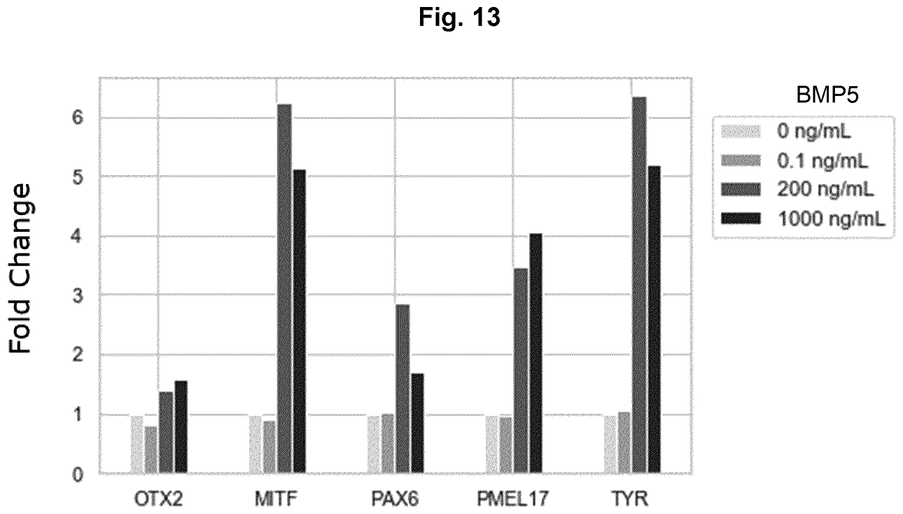

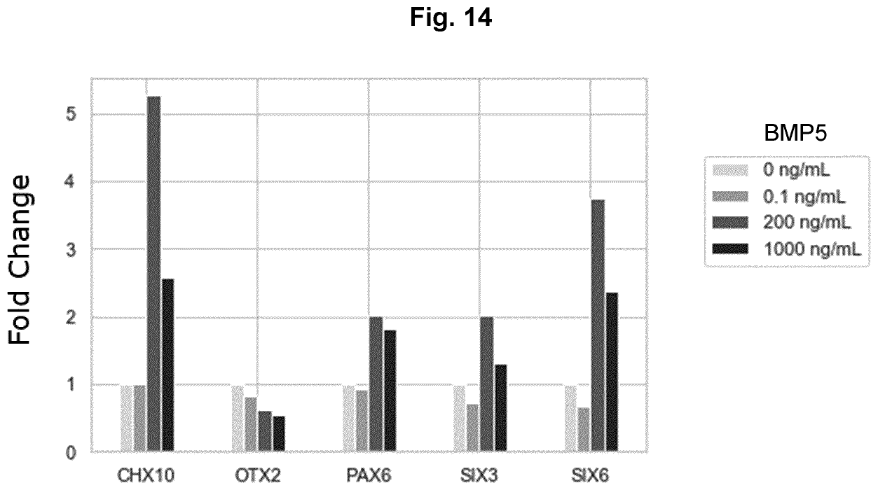

Here we summarize the tested conditions for FIGS. 13 and 14.

Condition: Single SMAD Inhibition and BMP5+CHIR99021 Treatment (FIG. 13)

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388

Day 7-11: 100% (GMEM+1% (v/v) N2+2% (v/v) B27)+GW788388 +BMP5

Day 12-21: 100% (GMEM +1% (v/v) N2+2% (v/v) B27)+BMP5+CHIR99021

Condition: Single SMAD Inhibition and BMP5 Treatment (FIG. 14)

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388

Day 7-11: 100% (GMEM+1% (v/v) N2+2% (v/v) B27)+GW788388+BMP5

Day 12-21: 100% (GMEM+1% (v/v) N2+2% (v/v) B27)+BMP5

As shown in FIG. 13, the best condition to induce eye field progenitor cells with a RPE progenitor cell identity was 200 ng/ml. As shown in FIG. 14, the best condition to generate eye field progenitor cells with a NR progenitor cell identity was also 200 ng/ml for NR progenitor cells, as evidenced by induction of VSX2 (also known as CHX10) gene expression.

In conclusion, of the concentrations tested, 200 ng/ml seems to be the best BMP5 concentration to generate eye field progenitor cells, and 1000 ng/ml also showed a positive but milder effect.

### Example 7

Protocol for Obtaining Eye Field Progenitor Cells Comparing Different BMPs

In order to investigate if BMP5 has a superior effect compared to other members of the BMP family generating eye field progenitor cells, we compared BMP5 to BMP4, BMP7, and to a BMP heterodimer formed by BMP4-BMP7, in combination with CHIR99021 to generate eye field progenitor cells with a RPE progenitor cell identity.

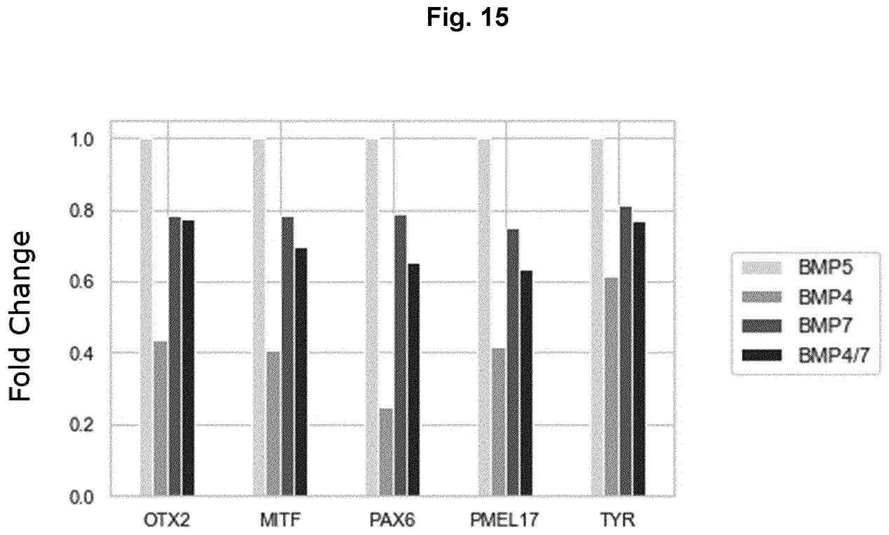

Here we summarize the tested condition for FIG. 15.

Condition: Single SMAD Inhibition and Different BMPs with CHIR99021

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388

Day 7-11: 100% (GMEM+1% (v/v) N2+2% (v/v) B27)+GW788388+BMP

Day 12-21: 100% (GMEM+1% (v/v) N2+2% (v/v) B27)+BMP+CHIR99021

As shown in FIG. 15, BMPS had a superior effect inducing gene expression of markers of eye field progenitor cells with a RPE progenitor cell identity. BMP4 showed the least capacity to induce these markers, and although BMP7 and the heterodimer BMP4-BMP7 were better than BMP4, the effect of BMPS was superior for all the markers showed here.

In conclusion, BMP5 has a superior effect on generating eye field progenitor cells compared to other BMP family members such as BMP4, BMP7 and the heterodimer BMP4-BMP7.

### Example 8

### Protocol for Obtaining Retinal Pigmented Epithelium (RPE) Progenitor Cells Using Dual SMAD Inhibition and Initial Inhibition of the WNT Pathway

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-6: 100% (GMEM+1% (v/v) N2+2% (v/v) B27)+NOGGIN+GW788388+Endo IWR1

Day 7-11: 100% (GMEM+1% (v/v) N2+2% (v/v) B27)+NOGGIN+GW788388+Endo IWR1+BMP5

Day 12-21: 100% (GMEM+1% (v/v) N2+2% (v/v) B27)+BMPS+CHIR99021

### Example 9

### Protocol for Obtaining Retinal Pigmented Epithelium (RPE) Progenitor Cells Using Single SMAD Inhibition and with Initial Inhibition of the WNT Pathway

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388+Endo IWR1

Day 7-11: 100% (GMEM+1% (v/v) N2+2% (v/v) B27)+GW788388+Endo IWR1+BMP5

Day 12-21: 100% (GMEM+1% (v/v) N2+2% (v/v) B27)+BMP5+CHIR99021

### Example 10

### Protocol for Obtaining Neural Retina Progenitor Cells Using Dual SMAD Inhibition and Initial Inhibition of the WNT Pathway

Day 0: 100% Nutristem+NOGGIN+GW788388+Y-27632

Day 1:50% Nutristem+50% (GMEM+1% (v/v) N2+2% (v/v) B27)+NOGGIN+GW788388

Day 2: 75% Nutristem+25% (GMEM+1% (v/v) N2+2% (v/v) B27)+NOGGIN+GW788388

Day 3-6: 100% (GMEM+1% (v/v) N2+2% (v/v) B27)+NOGGIN+GW788388+Endo IWR1

Day 7-11: 100% (GMEM+1% (v/v) N2+2% (v/v) B27)+NOGGIN+GW788388+Endo IWR1+BMP5

Day 12-21: 100% (GMEM+1% (v/v) N2+2% (v/v) B27)+BMP5

### Example 11

### Protocol for Obtaining Neural Retina Progenitor Cells Using Single SMAD Inhibition and with Initial Inhibition of the WNT Pathway

Day 0: 100% Nutristem+GW788388+Y-27632

Day 1: 50% Nutristem+50% (GMEM+1% (v/v) N2+2% (v/v) B27)+GW788388

Day 2: 75% Nutristem+25% (GMEM+1% (v/v) N2+2% (v/v) B27)+GW788388

Day 3-6: 100% (GMEM+1% (v/v) N2+2% (v/v) B27)+GW788388+Endo IWR1

Day 7-11: 100% (GMEM+1% (v/v) N2+2% (v/v) B27)+GW788388+Endo IWR1+BMP5

Day 12-21: 100% (GMEM+1% (v/v) N2+2% (v/v) B27)+BMP5

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for obtaining eye field progenitor cells from human pluripotent stem cells, comprising the steps of:
culturing the human pluripotent stem cells to obtain differentiating cells, and
contacting said differentiating cells with BMP5 (SEQ ID NO: 1) or an analog thereof, wherein said differentiating cells are allowed to differentiate into eye field progenitor cells.

2. The method according of claim 1, wherein said eye field progenitor cells are multiple progenitor cells of different cell lineages of the eye.

3. A method for obtaining eye field progenitor cells from human pluripotent stem cells, comprising the steps of:
seeding the human pluripotent stem cells on a substrate coated with a matrix,
culturing the human pluripotent stem cells in a cell culture medium to obtain differentiating cells,
contacting the differentiating cells with an inhibitor of Small Mothers Against Decapentaplegic (SMAD) protein signaling, and
contacting the differentiating cells with BMP5 (SEQ ID NO: 1) or an analog thereof,
wherein the differentiating cells are allowed to differentiate into eye field progenitor cells.

4. The method according to claim 1, wherein the differentiating cells are contacted with BMP5 (SEQ ID NO: 1).

5. The method according to claim 1, wherein the concentration of BMP5 is from about 0.1 ng/ml to about 2500 ng/ml, from about 100 ng/ml to about 500 ng/ml, from about 150 ng/ml to about 450 ng/ml, or from about 200 ng/ml to about 400 ng/ml.

6. The method according to claim 1, wherein the differentiating cells are contacted with an inhibitor of Small Mothers Against Decapentaplegic (SMAD) protein signaling selected from the group consisting of GW788388, LDN-193189, LY2157299, LY364947, NOGGIN, RepSOX, SB431542 and TEW-7197.

7. The method according to claim 6, wherein the inhibitor of Small Mothers Against Decapentaplegic (SMAD) protein signaling is GW788388 and/or RepSOX.

8. The method according to claim 3, wherein said matrix is a laminin or fragment thereof selected from the group consisting of laminin-511, laminin-521 and laminin-332, or a combination thereof.

9. The method according to claim 1, wherein the differentiating cells are contacted with BMP5 (SEQ ID NO: 1) or an analog thereof, from at about day 5 to at about day 15, from at about day 6 to at about day 12, from at about day 6 to at about day 8, or from about day 7.

10. The method according to claim 1, wherein the differentiating cells are contacted with an inhibitor of Small Mothers Against Decapentaplegic (SMAD) protein signaling from about day 0 to about day 15, to about day 14, to about day 13, or to about day 12.

11. The method according to claim 2, wherein the eye field progenitor cells are RPE progenitor cells, and wherein the method further comprises the step of:
contacting the differentiating cells with an inhibitor of GSK3.

12. The method according to claim 11, wherein the differentiating cells are contacted with the inhibitor of GSK3 from at about day 7 to at about day 15, or from about day 12.

13. The method according to claim 11, wherein the inhibitor of GSK3 is CHIR99021.

14. An in vitro cell population of eye field progenitor cells, wherein at least 40% of the eye field progenitor cells co-express PAX6 and OTX2, and at least one of VSX2 and/or MITF.

15. The in vitro cell population of eye field progenitor cells according to claim 14, wherein at least 50%, 60%, 70%, 80%, or 90% of the eye field progenitor cells co-express PAX6 and OTX2, and at least 10%, 20%, 30%, 40%, 50% of the eye field progenitor cells further co-express at least one of VSX2 and/or MITF.

16. The method according of claim 2, wherein said optic cup progenitor cells are selected from the group consisting of RPE progenitor cells and NR progenitor cells, lens progenitor cells and cornea progenitor cells.

17. The method according to claim 3, wherein the differentiating cells are contacted with BMP5 (SEQ ID NO: 1).

18. The method according to claim 3, wherein the concentration of BMPS is from about 0.1 ng/ml to about 2500 ng/ml, from about 100 ng/ml to about 500 ng/ml, from about 150 ng/ml to about 450 ng/ml, or from about 200 ng/ml to about 400 ng/ml.

19. The method according to claim 3, wherein the differentiating cells are contacted with an inhibitor of Small Mothers Against Decapentaplegic (SMAD) protein signaling selected from the group consisting of GW788388, LDN-193189, LY2157299, LY364947, NOGGIN, RepSOX, SB431542 and TEW-7197.

20. The method according to claim 19, wherein the inhibitor of Small Mothers Against Decapentaplegic (SMAD) protein signaling is GW788388 and/or RepSOX.

21. The method according to claim 3, wherein the differentiating cells are contacted with BMPS (SEQ ID NO: 1) or an analog thereof, from at about day 5 to at about day 15, from at about day 6 to at about day 12, from at about day 6 to at about day 8, or from about day 7.

22. The method according to claim 3, wherein the differentiating cells are contacted with an inhibitor of Small Mothers Against Decapentaplegic (SMAD) protein signaling from about day 0 to about day 15, to about day 14, to about day 13, or to about day 12.

23. The method according to claim 12, wherein the inhibitor of GSK3 is CHIR99021.


SEQUENCE LISTING (1 sequence)

SEQ ID NO: 1 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH